(19) United States
(12) Patent Application Publication
(10) Pub. No.: US 2025/0369872 A1
(43) Pub. Date: Dec. 4, 2025

(54) DETECTOR WITH TEMPERATURE DRIFT COMPENSATION

(71) Applicant: TRINAMIX GMBH, Ludwigshafen Am Rhein (DE)

(72) Inventors: Felix SCHMIDT, Ludwigshafen am Rhein (DE); Michael HANKE, Ludwigshafen am Rhein (DE)

(21) Appl. No.: 18/874,241

(22) PCT Filed: Jul. 13, 2023
(86) PCT No.: PCT/EP23/69472

§ 371 Date: Dec. 12, 2024

(30) Foreign Application Priority Data
Jul. 14, 2022 (EP) 22184879.9

(51) Int. Cl. G01N 21/27; G01J 1/42; G01N 21/31; G01N 21/359; G01N 21/47

(52) U.S. Cl. CPC G01N 21/274; G01J 1/42; G01N 21/314; G01N 21/359; G01N 21/4738; G01N 2201/1211; G01N 2201/1214; G01N 2201/1237

(57) ABSTRACT

Disclosed herein is a method for retrieving at least one alternating current (AC) signal S_{AC} from at least one measurement signal Sₘₑₐₛ of at least one detector. The measurement signal Sₘₑₐₛ includes the AC signal S_{AC} and at least one direct current (DC) signal S_{DC}. The AC signal S_{AC} has at least one predefined frequency f₀. The method includes the following steps:
a) monitoring the measurement signal Sₘₑₐₛ over time by using the detector;
b) determining the DC signal S_{DC} by using at least one evaluation unit; and
c) determining the AC signal S_{AC} by subtracting the DC signal S_{DC} from the measurement signal Sₘₑₐₛ by using the evaluation unit.

Also disclosed herein are a method for determining at least one item of information on at least one measurement object, a photodetector and a spectrometer.

### TECHNICAL FIELD

The invention relates to a method for retrieving at least one alternating current (AC) signal S_{AC} from at least one measurement signal Sₘₑₐₛ, a method for determining at least one item of measurement information on a measurement object, a photodetector and a spectrometer. Such methods and devices can, in general, be used for investigation or monitoring purposes, in particular in the infrared (IR) spectral region, especially in the near-infrared (NIR) spectral region, as well as for a detection of heat, flames, fire, or smoke. However, further kinds of applications are possible.

### BACKGROUND ART

Optical spectroscopic methods, specifically in the near-and mid-infrared spectral range, allow an insight into a molecular structure of an object by observing vibrations of molecular bonds. Such methods may for instance be used in spectroscopy, gas detection or thermometry. While mid-infrared light can be used to excite fundamental vibrational modes having high finesse and absorption strengths, the near-infrared spectral range can enable an observation of overtones and combination bands at lower absorption strengths. These advantages may enable to probe bulk objects and to obtain information on molecular constituents by using near-infrared spectroscopy. As a result, NIR spectroscopy can be widely applied in life and natural sciences, medicine, material science, agriculture, food, or pharmaceutical industries, e.g., for blood sugar measurements, pulse oximetry, fat content, material classification, product fraud identification, and many others.

However, providing analytical devices for the NIR wavelength range is, typically, rather difficult compared to spectrometers operating in visible light: Silicon-based light detectors are typically not applicable for light having a wavelength above 1.1 μm due to the band structure. However, indium, germanium, or lead salts or thermopiles can be applied. These materials can show strong dependencies on temperature. Besides temperature, other environmental effect may also contribute such as background light, stress or humidity. As a result, background signals may strongly drift at the time scale on environment drifts, such as in form of a direct current (DC) drift. A photo response, such as an alternating current (AC) signal may be extracted by using signal modulation, Fourier transform and lock in amplification schemes. However, retrieving the AC signal via Fourier analysis is typically also strongly affected by DC drifting due to the broadband Fourier spectrum of the DC contributions.

Typically, NIR detectors in laboratory spectrometers as well as in benchtop spectrometers are thermo-electrically cooled, often by using multiple stages, especially in order to achieve low temperatures, high detectivity and stabilization towards temperature drifts. However, thermo-electrical cooling, typically, yields technical complexity, size and power consumption, which impedes a wide-spread application of NIR spectroscopy, e.g. for point-of-care analytics, or in consumer devices. Therefore, operation of an IR spectrometer without cooling is desired, wherein the detector materials preferably function in a wide range of operation conditions and environment temperatures. As a result, temperature-induced drifts of the detector materials need to be compensated when comparing measurements to a reference signal or when repeating measurements in order to reduce measurement noise.

In the prior art, devices and methods are known, which apply a temperature correction based on a temperature sensor, or based on a second optical detector which is identical to the primary detector.

JP H01110225 A discloses a stable infrared radiation meter without use of a mechanical part such as a chopper, implemented by monitoring a temperature of an optical system to compensate for a temperature drift at a zero point. A detector comprising a photodiode, such as InSb and HgCdTe, is placed into a vacuum container and cooled by liquid nitrogen. Infrared rays from a measuring point form an image on a light detecting surface of the detector. A field of view of the detector is restricted by a cold shield. Temperature of an optical system is monitored by a temperature sensor to compensate for a temperature drift at the zero point of the infrared radiation meter using an output thereof.

CN 2359677 Y discloses an infrared optical fiber temperature measuring device used for smelting and casting. The infrared optical fiber temperature measuring device comprises a positioning cylinder, a hemispherical reflector, a focusing object lens, an optical fiber bundle, a filter, a detector and a temperature compensation circuit, wherein, the positioning cylinder nears the surface of melting liquid steel, and the hemispherical reflector buckles one end of the positioning cylinder above the surface of the liquid steel to be measured; the focusing object lens is installed at the top of the hemispherical reflector, one end surface of the optical fiber bundle is installed in the focal length position of the focusing objective lens, and the other end surface is coupled with the detector through the filter; the output end of the detector is connected with the temperature compensation circuit.

U.S. Pat. No. 6,852,966 B1 discloses a method and apparatus for compensating a photo-detector allowing both regulation and monitoring of the photo-detector to be performed with a common digital controller. The controller accepts input of monitored operational parameters including received signal strength and temperature. The controller provides as an output a bias control signal which regulates a positive or negative side bias voltage power supply for the photo-detector. The controller maintains the bias voltage to the photo-detector at levels. The controller includes a corresponding digital signal strength and temperature compensators the outputs of which summed with a summer to provide the bias control signal. The digital signal strength compensator also provides as an output a monitor signal a level of which corresponds to the actual signal strength received by the photo-detector after compensation for the variable gain of the photo-detector resulting from the bias voltage level. A transceiver as well as methods and means for monitoring a photo-detector are also disclosed.

US 20110255075 A1 discloses a spectrometric assembly and method for determining a temperature value for a detector of a spectrometer. It is conventional to record the detector temperature in an optoelectronic detector using a thermal temperature sensor in order to compensate for temperature fluctuations. Due to the finite distance between the detector and the temperature sensor, the accuracy of the temperature detection is limited. In addition to means for spectral division of incident tight and an optical detector for spectrally resolved detection of a spectral range of the divided light, a second optical detector is provided for detection of a partial range of this spectral range as a reference detector.

CN 109307550 A discloses a temperature compensation method for temperature compensation of optical power meters. The temperature compensation method comprises the following steps of: placing the optical power meter in a high-low temperature chamber, sequentially adjusting the temperature from −10° C. to 40° C., recording the zero point value of different gears at each temperature point by a CPU module, and calculating the zero drift of the current gear caused by the temperature difference according to a reference temperature; and obtaining the optical power value detected by a photoelectric detector by the CPU module, setting a current optical power detection gear, obtaining the real-time temperature detected by a temperature sensor, and sending the calibration factor of the zero drift to a secondary amplification circuit through the temperature compensation circuit by the CPU module according to the temperature drift generated by the reference temperature in the current gear for hardware circuit compensation.

JP S61213650 A discloses optical measuring equipment. Radiation energy light from an object to be measured is converged by a lens and stopped down by a slit and made to parallel rays by a lens. Then, the light is spectroscopically separated by a spectroscope and is made incident onto each element of a detector as light of different wavelength. Gradient or function of the rate of variation of spectral sensitivity of measuring wavelength of each element of the detector is stored beforehand in a memory. Temperature T of the detector is detected by a temperature sensor at the time of measuring, and output of each element of the detector is calculated and corrected by an arithmetic unit basing on gradient or function of each element of the memory and temperature T of the sensor.

CN 103076087 A discloses a mid-infrared photoelectric detector driving circuit, a detector assembly and a detector assembly array.

DE 102009026951 A1 discloses a spectroscopic gas sensor with an infrared source, an absorption chamber, an optical filter and a detector with a detector element forming a measuring beam from the infrared source through the absorption chamber and the optical filter to the detector. The detector element is arranged in the measuring beam and generates a measuring signal.

The detector is a pyroelectric detector with an internal temperature compensation device which generates a temperature-compensated result signal from the measurement signal.

US 2019/317016 A1 describes an analyzer for identifying or verifying or otherwise characterizing a liquid based drug sample comprising: an electromagnetic radiation source for emitting electromagnetic radiation in at least one beam at a sample, the electromagnetic radiation comprising at least two different wavelengths, a sample detector that detects affected electromagnetic radiation resulting from the emitted electromagnetic radiation affected by the sample, and a processor for identifying or verifying the sample from the detected affected electromagnetic radiation, wherein each wavelength or at least two of the wavelengths is between substantially 1300 nm and 2000 nm, and each wavelength or at least two of the wavelengths is in the vicinity of the wavelength(s) of (or within a region spanning) a spectral characteristic in the liquid spectrum between substantially 1300 nm and 2000 nm.

WO 2014/054022A1 describes an analyser for characterising a sample comprising: an integrated laser for emitting electromagnetic radiation at least one beam along a single-mode (SM) and polarization maintaining (PM) channel at a sample, the electromagnetic radiation comprising at least two different wavelengths, a sample detector that detects affected electromagnetic radiation resulting from the emitted electromagnetic radiation affected by the sample and provides output representing the detected affected radiation, and a processor for characterizing the sample from the detector output representing the detected affected electromagnetic radiation.

Despite the advantages as implied by the above-mentioned devices and methods, there still is a need for improvements. Specifically, additional sensors or detectors are required for compensating drifts, such as temperature-induced drifts, of detector signals, which adds to the cost, to the complexity and thus also to the susceptibility to errors of the devices and methods. As an example, the additional detectors may fail or they may drift themselves, specifically in a different fashion compared to a primary detector.

### Problem to be Solved

It is therefore desirable to provide methods and devices for compensating measurement signals which at least substantially avoid the disadvantages of known methods and devices of this type. In particular, it is desirable to provide methods and devices which ensure an accurate and reliable compensation of drifts of detectors, specifically of temperature drifts of photodetectors, in a simple and safe fashion, specifically without the need of installing additional components.

### SUMMARY

This problem is addressed by the invention with the features of the independent claims. Advantageous embodiments which might be realized in an isolated fashion or in any arbitrary combinations are listed in the dependent claims as well as throughout the specification.

In a first aspect of the present invention, a method for retrieving at least one alternating current (AC) signal S_{AC} from at least one measurement signal Sₘₑₐₛ of at least one detector is disclosed. The measurement signal Sₘₑₐₛ comprises the AC signal S_{AC} and at least one direct current (DC) signal S_{DC}. The AC signal S_{AC} has at least one predefined frequency f₀. The method comprises the following steps:
a) monitoring the measurement signal Sₘₑₐₛ over time by using the detector;
b) determining the DC signal S_{DC} by using at least one evaluation unit, wherein the determining comprises evaluating the measurement signal Sₘₑₐₛ by using at least one of the frequency f₀ and at least one overtone of the frequency f₀; and
c) determining the AC signal S_{AC} by subtracting the DC signal S_{DC} from the measurement signal Sₘₑₐₛ by using the evaluation unit.

The method steps may be performed in the indicated order. It shall be noted, however, that a different order is also possible. The method may comprise further method steps which are not listed. Further, one or more of the method steps may be performed once or repeatedly. Further, two or more of the method steps may be performed simultaneously or in a timely overlapping fashion.

The method may comprise correcting at least one environmental change affecting the measurement signal Sₘₑₐₛ. The environmental change may specifically comprise at least one of a temperature change, a change in a background light, a mechanical stress, and a humidity change and a degradation of at least a part of the detector. The term “correcting” including any grammatical variation thereof as used herein is a broad term and is to be given its ordinary and customary meaning to a person of ordinary skill in the art and is not to be limited to a special or customized meaning. The term specifically may refer, without limitation, to compensating or readjusting an entity. The correcting may comprise removing or eliminating perturbations, specifically external perturbations, affecting the measurement signal Sₘₑₐₛ. Specifically, the correcting may comprise removing a contribution to the measurement signal Sₘₑₐₛ caused by an environmental change, such as a temperature change. Such a contribution may refer to a DC signal S_{DC}. As an example, the detector may be a photodetector of a spectrometer configured for measuring optical radiation. Other external influences besides the optical radiation to be measured may not be of interest in the measurement and may only disturb the measurement signal Sₘₑₐₛ. The spectrometer may further comprise a modulated radiation source. Thus, the signal of interest may be an AC signal S_{AC}. External influences, e.g. temperature, may typically change on larger time scales compared to the AC signal S_{AC} and may be one-directional at least in the monitored period of time. The external influences may typically contribute in form of a DC signal S_{DC} to the measurement signal Sₘₑₐₛ. Identifying the DC signal S_{DC} in the measurement signal Sₘₑₐₛ and removing the DC signal S_{DC} from the measurement signal Sₘₑₐₛ may thus lead to the AC signal S_{AC} which may be of particular interest in the measurement. Further options are feasible.

Known methods, such as described in US 2019/317016 A1 and WO 2014/054022A1, propose removing a dark current component using a reference and a sample detector, Fourier Transformation and Fourier analysis. In contrast, the present invention proposes using the measurement signal only and using the frequency and/or at least one overtone of the frequency to determine a DC component, as described in steps b) and c). Possible options for determining the DC component are described in more detail below.

The term “retrieving” including any grammatical variation thereof as used herein is a broad term and is to be given its ordinary and customary meaning to a person of ordinary skill in the art and is not to be limited to a special or customized meaning. The term specifically may refer, without limitation, to at least one of determining, deriving and filtering out a signal or at least a part of the signal. As said, the measurement signal Sₘₑₐₛ comprises the AC signal S_{AC} and the DC signal S_{DC}. The retrieving may comprise identifying and/or isolating the AC signal S_{AC} in the measurement signal Sₘₑₐₛ. The retrieving may comprise removing and/or eliminating the DC signal S_{DC} from the measurement signal Sₘₑₐₛ. The retrieving may comprise providing the AC signal S_{AC} to further entities for further processing and/or evaluation, such as for determining an item of information, e.g. on at least one measurement object.

The term “signal” as used herein is a broad term and is to be given its ordinary and customary meaning to a person of ordinary skill in the art and is not to be limited to a special or customized meaning. The term specifically may refer, without limitation, to an observable change in at least one physical quantity. The signal may be or comprise a sign or a function conveying information about the at least one physical quantity. The signal may specifically be or comprise at least one of an electronic signal, an optical signal or an optoelectronic signal. The signal may be a variable signal, specifically over time. The signal may be an analog signal. The signal may be or comprise at least one of a variable voltage, a variable current, a variable charge, a variable resistance or, generally, a variable electromagnetic wave. The variable electromagnetic wave may comprise at least one of a variable amplitude, a variable frequency or a variable phase. The signal may be a digital signal. The signal may comprise at least one count. Further options are feasible. The signal may specifically be related to at least one measurement. The signal may specifically be generated by the at least one detector.

The term “measurement signal” as used herein is a broad term and is to be given its ordinary and customary meaning to a person of ordinary skill in the art and is not to be limited to a special or customized meaning. The term specifically may refer, without limitation, to a signal relating to at least one measurement, more specifically to at least one measurement object. The measurement signal may be a signal generated by a detector upon detection of at least one physical quantity, such as a physical quantity of a measurement object. The measurement signal may comprise at least one electronic signal, such as a current or a voltage or a resistance. The measurement signal may comprise an analog signal. The measurement signal may comprise a digital signal, such as a count. The measurement signal may be a superposition of two or more signals or sub-signals. The measurement may be affected by plurality of influences, such as illumination, temperature, humidity or mechanical stress. Each influence may contribute to the measurement signal. The measurement signal may be dividable into two or more sub-signals, wherein the sub-signals may at least partially relate to different influences.

The measurement signal Sₘₑₐₛ comprises the AC signal S_{AC} and at least one direct current (DC) signal S_{DC}. The term “direct current signal”, abbreviated by DC signal, as used herein is a broad term and is to be given its ordinary and customary meaning to a person of ordinary skill in the art and is not to be limited to a special or customized meaning. The term specifically may refer, without limitation, to a one-directional or at least essentially one-directional signal over time, such as a continuously increasing signal over time or a continuously decreasing signal over time. As an example, the DC signal S_{DC} may be a digital signal, wherein a count may continuously increases over time. The DC signal S_{DC} may comprise at least one plateau over the course of time. Deviations from a strictly one-directional progression may e.g. arise due to signal noise or external perturbations.

The term “alternating current signal”, abbreviated by AC signal, as used herein is a broad term and is to be given its ordinary and customary meaning to a person of ordinary skill in the art and is not to be limited to a special or customized meaning. The term specifically may refer, without limitation, to a signal which over time reverses direction and/or changes its magnitude, e.g. periodically. As an example, the AC signal S_{AC} may be a digital signal, wherein a count increases and decreases over time in an alternating fashion. The AC signal S_{AC} may be a sinusoidal signal, a square wave, a pulse-width modulated signal, or a combination of the previously mentioned ones. The AC signal S_{AC} may be a periodic signal or an at least essentially periodic signal. Deviations from a strictly periodic progression may e.g. arise due to signal noise or external perturbations. As said, the AC signal S_{AC} has at least one predefined frequency f₀.

The term “frequency” as used herein is a broad term and is to be given its ordinary and customary meaning to a person of ordinary skill in the art and is not to be limited to a special or customized meaning. The term specifically may refer, without limitation, to a number of occurrences of a repeating event over time. The frequency can be defined as a reciprocal of a period duration, such as a period duration of a periodic signal. The frequency may be predefined by at least one default, such as at least one default in a measurement setup. A user may be allowed to set the default or to choose between a number of different available defaults. As an example, which will also be described in further detail below, the detector may be a photodetector of a spectrometer, wherein the spectrometer may further comprise a modulated radiation source. Thus, the frequency of the AC signal S_{AC} may be predefined by setting a specific modulation frequency at the modulated radiation source. Other options are feasible.

A frequency, such as the frequency f₀, generally has a plurality of overtones. The term “overtone” as used herein is a broad term and is to be given its ordinary and customary meaning to a person of ordinary skill in the art and is not to be limited to a special or customized meaning. The term specifically may refer, without limitation, to a frequency which is a harmonic of a fundamental frequency, such as of the frequency f₀. An overtone of the frequency f₀ may be a positive integer multiple of the frequency f₀, such as 2 f₀, 3 f₀, 4 f₀ and so on.

The term “detector” as used herein is a broad term and is to be given its ordinary and customary meaning to a person of ordinary skill in the art and is not to be limited to a special or customized meaning. The term specifically may refer, without limitation, to a measurement device, such as a sensor, configured for generating at least one measurement signal. The detector may be configured for sensing or detecting or monitoring at least one physical quantity. The detector may be an electronic device or an optoelectronic device. The detector may be configured for generating at least one electronic signal, such as a current or a voltage or a resistance. The detector may specifically be or comprise a photodetector as will be described in further detail below. However, other kinds of detectors are also feasible.

The detector may comprise at least one photodetector. The photodetector may comprise at least one photosensitive region. Step a) may comprise measuring the measurement signal Sₘₑₐₛ by using the photosensitive region of the photodetector. The measurement signal Sₘₑₐₛ may be dependent on an illumination of the photosensitive region. The term “photodetector” as used herein is a broad term and is to be given its ordinary and customary meaning to a person of ordinary skill in the art and is not to be limited to a special or customized meaning. The term specifically may refer, without limitation, to an optical detector or optical sensor configured for detecting optical radiation, such as for detecting an illumination and/or a light spot generated by at least one light beam. The photodetector may comprise at least one substrate. A single photodetector may be a substrate with at least one single photosensitive area, which generates a physical response to the illumination for a given wavelength range.

The photodetector may comprise at least one photosensitive region. The photodetector may comprise a plurality of photosensitive regions, which may be arranged in at least one of an array or a matrix. The term “photosensitive region” as used herein is a broad term and is to be given its ordinary and customary meaning to a person of ordinary skill in the art and is not to be limited to a special or customized meaning. The term specifically may refer, without limitation, to a unit of a photodetector configured for being illuminated, or in other words for receiving optical radiation, and for generating at least one signal, such as an electronic signal, in response to the illumination. The photosensitive region may be located on a surface of the photodetector. The photosensitive region may specifically be a single, closed, uniform photosensitive region. However, other options may also be feasible. The photosensitive region may also be referred to as pixel.

The term “illumination” as used herein is a broad term and is to be given its ordinary and customary meaning to a person of ordinary skill in the art and is not to be limited to a special or customized meaning. The term specifically may refer, without limitation, to optical radiation, specifically within at least one of the visible, the ultraviolet or the infrared spectral range. The term “ultraviolet”, generally, refers to electromagnetic radiation having a wavelength of 1 nm to 380 nm, preferably of 100 nm to 380 nm. Further, the term “visible”, generally, refers to a wavelength of 380 nm to 760 nm. Further, the term “infrared”, “abbreviated to IR”, generally refers to a wavelength of 760 nm to 1000 μm, wherein the wavelength of 760 nm to 3 μm is, usually, denominated as “near infrared”, abbreviated to “NIR”. Preferably, the illumination which is used for typical purposes of the present invention is IR radiation, more preferred, NIR radiation, especially of a wavelength of 760 nm to 3 μm, preferably of 1 μm to 3 μm. The illumination may specifically be optical radiation impinging the photodetector, or more specifically the photosensitive region. The term “illumination” may also be referred to as “optical radiation” or as “light” herein.

The illumination may be provided by at least one measurement object, wherein the providing may comprise at least one of a reflecting, transmitting and emitting. Specifically, before interacting with the measurement object, the illumination may e.g. be emitted by at least one radiation source. The term “radiation source” as used herein is a broad term and is to be given its ordinary and customary meaning to a person of ordinary skill in the art and is not to be limited to a special or customized meaning. The term specifically may refer, without limitation, to a device configured for emitting optical radiation. The radiation source may be configured for emitting optical radiation towards the measurement object, such as in form of a light beam. The radiation source may be configured for isotopically emitting optical radiation, e.g. uniformly in all spatial directions, wherein only a part of the emitted optical radiation may impinge the measurement object. The radiation source may comprise at least one of a semiconductor-based radiation source or a thermal radiator. The at least one semiconductor-based radiation source may be selected from at least one of a light emitting diode (LED) or a laser, specifically a laser diode. The LED may comprise at least one fluorescent and/or phosphorescent material. The thermal radiator may comprise at least one of an incandescent lamp, a black body emitter and a microelectromechanical system (MEMS) emitter. Further kinds of radiation sources may also be feasible.

The illumination may be modulated, e.g. by using a modulated radiation source. The radiation source may be a modulated radiation source. The radiation source may be modulated at the frequency f₀. Thus, the frequency f₀ and overtones of the frequency f₀ may be present in the optical radiation impinging the photodetector and subsequently also in the measurement signal Sₘₑₐₛ as generated by the photodetector. Specifically, as said, the AC signal S_{AC} has the frequency f₀. The term “modulating” including any grammatical variation thereof as used herein is a broad term and is to be given its ordinary and customary meaning to a person of ordinary skill in the art and is not to be limited to a special or customized meaning. The term specifically may refer, without limitation, to a process of changing, specifically periodically changing, at least one property of optical radiation, specifically one or both of an intensity or a phase of the optical radiation. As the skilled person will know, the intensity again relates to an amplitude of the optical radiation. The modulation may be a full modulation from a maximum value to zero, or may be a partial modulation, from a maximum value to an intermediate value greater than zero. The modulating may comprise using a modulating element. The modulating element may be configured for e.g. mechanically modulating the optical radiation, e.g. by using a rotating chopper wheel, and/or for electronically modulating the optical radiation, e.g. by using an electrooptic effect and/or an acoustoptic effect, e.g. by using a Pockels cell and/or a Kerr cell. Further options are feasible.

The photodetector may be configured for detecting optical radiation in a wavelength of 300 nm to 3000 nm, specifically 500 nm to 2500 nm, more specifically 1400 nm to 2000 nm. The photosensitive region may comprise at least one photoconductive material. The photoconductive material may be selected from at least one of PbS, PbSe, Ge, InGaAs, InSb, and HgCdTe. Other options, such as photodiodes or thermopiles, may also be feasible. The photodetector may be configured for generating at least one measurement signal, specifically in response to an illumination of the photosensitive region, such as a photocurrent. However, besides the illumination, the measurement signal may also be subject to other influences, as the skilled person will already know. Specifically, environmental changes, such as temperature changes, may also affect the measurement signal and may more specifically lead to a drift in the measurement signal.

Step a) comprises monitoring the measurement signal Sₘₑₐₛ over time by using the detector. The term “monitoring over time” including any grammatical variation thereof as used herein is a broad term and is to be given its ordinary and customary meaning to a person of ordinary skill in the art and is not to be limited to a special or customized meaning. The term specifically may refer, without limitation, to at least one of measuring, observing or recording an entity, such as the measurement signal Sₘₑₐₛ, specifically over time. The monitoring may comprise recording a progression and/or a development of the measurement signal Sₘₑₐₛ over time.

Step b) comprises determining the DC signal S_{DC} by using at least one evaluation unit, wherein the determining comprises evaluating the measurement signal Sₘₑₐₛ by using at least one of the frequency f₀ and at least one overtone of the frequency f₀. The term “evaluating” including any grammatical variation thereof as used herein is a broad term and is to be given its ordinary and customary meaning to a person of ordinary skill in the art and is not to be limited to a special or customized meaning. The term specifically may refer, without limitation, to processing or analyzing or interpreting an entity, such as the measurement signal Sₘₑₐₛ. The evaluating may comprise performing at least one mathematical calculation involving the measurement signal Sₘₑₐₛ. The evaluating may comprise transforming and/or converting the measurement signal Sₘₑₐₛ. The evaluating may comprise using at least one relationship, such as a predefined and/or predetermined relationship, e.g. from a look-up table, or a variable relationship, such as function. The evaluating may comprise filtering and/or smoothening the measurement signal Sₘₑₐₛ. The evaluating may comprise deriving at least one qualitative or quantitative item of information from the measurement signal Sₘₑₐₛ, such as a contribution of the DC signal S_{DC} and/or the AC signal S_{AC} to the measurement signal Sₘₑₐₛ. Different approaches may be possible for such purpose as will be outlined in further detail below.

The term “evaluation unit” as used herein is a broad term and is to be given its ordinary and customary meaning to a person of ordinary skill in the art and is not to be limited to a special or customized meaning. The term specifically may refer, without limitation, to a device configured for analyzing or interpreting data, specifically for determining at least one item of qualitative or quantitative information. The information may specifically be obtained by evaluating at least one signal, such as a signal generated by the detector, specifically the measurement signal Sₘₑₐₛ.

The evaluation unit may be or may comprise at least one of an integrated circuit, in particular an application-specific integrated circuit (ASIC), or a data processing device, in particular at least one of a digital signal processor (DSP), a field programmable gate array (FPGA), a microcontroller, a microcomputer, a computer, or an electronic communication unit, specifically a smartphone or a tablet. Further components may be feasible, in particular at least one preprocessing device or data acquisition device. The evaluation unit may comprise at least one interface, in particular at least one of a wireless interface or a wire-bound interface. The evaluation unit can be designed to, completely or partially, control or drive further devices, such as the detector. The evaluation unit may be designed to carry out at least one measurement cycle in which a plurality of measurement signals may be picked up. The evaluation unit may be designed to control the detector for performing at least one measurement and/or for generating at least one measurement signal.

Information as determined by the evaluation unit may, in particular, be provided to at least one of a further apparatus, or to a user, preferably in at least one of an electronic, visual, acoustic, or tactile fashion. The information may be stored in at least one data storage unit, specifically in an internal data storage unit as comprised by the photodetector, in particular by the at least one evaluation unit, or in an separate storage unit to which the information may be transmitted via the at least one interface. The separate storage unit may be comprised by the at least one electronic communication unit. The storage unit may in particular be configured for storing at least one electronic table, such as at least one look-up table.

The evaluation unit may be configured to perform at least one computer program, in particular at least one computer program performing or supporting the step of generating the at information. By way of example, one or more algorithms may be implemented which, by using the at least one measurement signal as at least one input variable, may perform a transformation into a piece of information. For this purpose, the evaluation unit may comprise at least one data processing device, in particular at least one of an electronic or an optical data processing device, which can be designed to generate the information by evaluating the at least one measurement signal. The evaluation unit may be designed to use at least one measurement signal as at least one input variable and to generate the information by processing the at least one input variable. The processing can be performed in a consecutive, a parallel, or a combined manner. The evaluation unit may use an arbitrary process for generating the information, in particular by calculation and/or using at least one stored and/or known relationship.

The detector may comprise the evaluation unit and/or at least one interface for transmitting data from and/or to and/or within the evaluation unit. The term “interface” as used herein is a broad term and is to be given its ordinary and customary meaning to a person of ordinary skill in the art and is not to be limited to a special or customized meaning. The term specifically may refer, without limitation, to an item or element forming a boundary configured for transferring information. The interface may specifically be a communication interface. In particular, the interface may be configured for transferring information from a computational device, e.g. a computer, such as to send or output information, e.g. onto another device. Additionally or alternatively, the interface may be configured for transferring information onto a computational device, e.g. onto a computer, such as to receive information. The interface may specifically provide means for transferring or exchanging information. In particular, the interface may provide a data transfer connection, e.g. Bluetooth, NFC, inductive coupling or the like. As an example, the interface may be or may comprise at least one port comprising one or more of a network or internet port, a USB-port and a disk drive. The interface may comprise at least one web interface.

The evaluation unit may at least partially be cloud based. The term “cloud based” as used herein is a broad term and is to be given its ordinary and customary meaning to a person of ordinary skill in the art and is not to be limited to a special or customized meaning. The term specifically may refer, without limitation, to an outsourcing of the evaluation unit or of parts of the evaluation unit to at least partially interconnected external devices, specifically computers or computer networks having larger computing power and/or data storage volume. The external devices may be arbitrarily spatially distributed. The external devices may vary over time, specifically on demand. The external devices may be interconnected by using the internet and/or at least one intranet. The external devices may each comprise at least one interface, such as a communication interface for transferring data.

As indicated, a plurality of specific approaches may exist for determining the DC signal S_{DC} by using at least one of the frequency f₀ and at least one overtone of the frequency f₀ in step b). As an example, which will also be outlined in further detail below, the measurement signal Sₘₑₐₛ may be transformed into a frequency domain for filtering for the frequency f₀ and/or at least one overtone of the frequency f₀. The filtered transformed measurement signal Sₘₑₐₛ may then be used for determining the DC signal S_{DC}, such as by fitting the DC signal S_{DC} to the filtered transformed measurement signal Sₘₑₐₛ. In the following, specific approaches for determining the DC signal S_{DC} will be presented. The approaches may be performed alternatively or additionally, such as successively. Further approaches may exist and may be used for determining the DC signal S_{DC}.

The DC signal S_{DC} may be determined by further using a phase q of the measurement signal Sₘₑₐₛ, specifically of the AC signal S_{AC}, specifically besides the frequency f₀. The evaluation of the measurement signal Sₘₑₐₛ may comprise determining local minima of the measurement signal Sₘₑₐₛ by using the frequency f₀ and the phase φ and at least one of the frequency f₀ and at least one overtone of the frequency f₀. The DC signal S_{DC} may be determined by using the local minima. The term “phase” as used herein is a broad term and is to be given its ordinary and customary meaning to a person of ordinary skill in the art and is not to be limited to a special or customized meaning. The term specifically may refer, without limitation, to a position indicator within a periodic signal. Typically, the phase may be represented as angle as the skilled person will know. The phase may be dependent on the time, on a frequency of the signal and/or on a phase offset. Specifically, the phase may indicate when a periodic signal or a periodic part of a total signal, such as a periodic sub-signal, reaches an extremum, such as a minimum or a maximum. As an example, the measurement signal Sₘₑₐₛ may show an at least essentially periodic sub-signal, e.g. due to using a modulated radiation source as already outlined. This sub-signal may correspond to the AC signal S_{AC} which may be of interest in the end, but which at this stage may drift e.g. due to an environmental change. The frequency f₀ and the phase φ of this sub-signal may be used for identifying minima in the sub-signal, which may simultaneously at least be local minima in the overall measurement signal Sₘₑₐₛ.

The term “local minimum” as used herein is a broad term and is to be given its ordinary and customary meaning to a person of ordinary skill in the art and is not to be limited to a special or customized meaning. The term specifically may refer, without limitation, to a lowest signal value in a signal interval, specifically within a period of a periodic signal. The measurement signal Sₘₑₐₛ may specifically be recorded in counts over time. As said, the measurement signal comprises the AC signal S_{AC} and the DC signal S_{DC}. A local minimum may be a lowest signal value, such as a lowest count, in a time interval relating to a period of the AC signal S_{AC}. Contrarily, as the skilled person will know, a global minimum may be the lowest signal value over the entire signal. The signal may comprise a plurality of local minima. The local minima may at least partially have the same level. The local minima may specifically at least partially have different levels, specifically due to external perturbations, such as an environmental change affecting the detector and/or the measurement signal.

The evaluation of the measurement signal Sₘₑₐₛ may comprise fitting the DC signal S_{DC} to the local minima of the measurement signal Sₘₑₐₛ. The DC signal S_{DC} may be a function S_{DC}(t) over time comprising at least one of a polynomial function having at least one fit parameter, an exponential function having at least one fit parameter, a square root function having at least one fit parameter and a logarithmic function having at least one fit parameter. The term “fitting” including any grammatical variation thereof as used herein is a broad term and is to be given its ordinary and customary meaning to a person of ordinary skill in the art and is not to be limited to a special or customized meaning. The term specifically may refer, without limitation, to a regression analysis for estimating a relation between at least two variables. The fitting may comprise at least one of a linear regression, a partial least square regression, a non-linear regression, an interpolation and an extrapolation. The fitting may comprise at least one regression model, e.g. a trained model. The fitting may comprise using at least one fit function, such as at least one of the functions listed above. The function S_{DC}(t) may be a fit function fitted to the local minima of the measurement signal Sₘₑₐₛ. The fit function may comprise at least one fit parameter. The term “fit parameter” as used herein is a broad term and is to be given its ordinary and customary meaning to a person of ordinary skill in the art and is not to be limited to a special or customized meaning. The term specifically may refer, without limitation, to a parameter or coefficient of a fit function. As an example, the fit function may be a linear function and the fit parameters may be a slope and an offset of the linear function. Generally, a variety of further options is feasible and known to the skilled person.

Additionally or alternatively to the above-described approach using the local minima of the measurement signal Sₘₑₐₛ, in step b) the DC signal S_{DC} may be determined by transforming the measurement signal Sₘₑₐₛ into a frequency domain. The term “frequency domain” as used herein is a broad term and is to be given its ordinary and customary meaning to a person of ordinary skill in the art and is not to be limited to a special or customized meaning. The term specifically may refer, without limitation, to an analysis of a signal with respect to at least one frequency of the signal. A signal may typically be recorded over time in the time domain meaning that a signal value is related to a specific point in time. However, as the skilled person will know, for a variety of applications, it may be helpful to analyze the signal with respect to frequencies comprised by the signal in the frequency domain. As an example, one total signal may comprise a plurality of sub-signals each comprising a specific frequency. The sub-signals may be distinguishable, e.g. for further isolated processing, by analyzing the frequencies in the total signal. In the frequency domain, a signal value may be related to a specific frequency. Signal values, such as signal values of a total signal, may be plotted over a frequency interval in the frequency domain.

The measurement signal Sₘₑₐₛ may be transformed into the frequency domain by using a Fourier transformation. The frequency domain may also be referred to as Fourier space. The term “Fourier transformation” as used herein is a broad term and is to be given its ordinary and customary meaning to a person of ordinary skill in the art and is not to be limited to a special or customized meaning. The term specifically may refer, without limitation, to an integral transformation for decomposing an integratable function depending on space or time into a function depending on spatial frequency or temporal frequency. Typically, the temporal frequency is simply referred to as frequency herein. Specifically, the Fourier transformation may be configured for decomposing a time dependent signal into a frequency dependent signal. The Fourier transformation may comprise at least one of a Fourier analysis, a continuous Fourier transformation, a discrete Fourier transformation and a Fourier related transformation, such as a Laplace transformation for instance. Further options are feasible. Generally, there are several common and well known conventions for defining or performing a Fourier transformation of a function, such as a fast Fourier transformation, and also for defining a corresponding inverse Fourier transformation. Further transformations, besides a Fourier transformation, may also be applicable for the present invention. In step b), the DC signal S_{DC} may specifically be determined by setting at least one of the frequency f₀ and at least one overtone of the frequency f₀ to zero in the frequency domain before retransformation into the time domain, such as by using an inverse Fourier transformation. Thus, step c) may specifically be performed in the time domain again.

The evaluation of the measurement signal Sₘₑₐₛ, such as performed in step b), may comprise filtering the transformed measurement signal Sₘₑₐₛ for at least one of the frequency f₀ and at least one overtone of the frequency f₀. The term “filtering” including any grammatical variation thereof as used herein is a broad term and is to be given its ordinary and customary meaning to a person of ordinary skill in the art and is not to be limited to a special or customized meaning. The term specifically may refer, without limitation, to selectively extracting at least one part of a signal, such as a specific signal interval, e.g. in the frequency domain. The filtering may comprise extracting specific frequencies, or sub-signals having specific frequencies, out of a total signal in the frequency domain. Specifically, the filtering may comprise extracting the at least one of the frequency f₀ and at least one overtone of the frequency f₀ from the measurement signal Sₘₑₐₛ. The rest of the original measurement signal Sₘₑₐₛ may stay as a remainder. The frequency f₀ and overtones of the frequency f₀ may refer to the AC signal S_{AC}. The remainder may refer to the DC signal S_{DC}. The filtering may comprise using at least one electronic filter element, specifically at least one frequency electronic filter element, such as an electronic bandpass filter element. The electronic filter element may be an analog electronic filter element or a digital electronic filter element. Other options are feasible and generally known to the skilled person.

The evaluation of the measurement signal Sₘₑₐₛ may comprise using the filtered transformed measurement signal Sₘₑₐₛ for determining the DC signal S_{DC}. Specifically, the evaluation of the measurement signal Sₘₑₐₛ may comprise using a remainder of the filtering of the transformed measurement signal Sₘₑₐₛ for determining the DC signal S_{DC} The evaluation of the measurement signal Sₘₑₐₛ may comprise fitting the DC signal S_{DC} to the filtered transformed measurement signal Sₘₑₐₛ, specifically to the remainder of the filtering of the transformed measurement signal Sₘₑₐₛ. The DC signal S_{DC} may be a function S_{DC}(t) over time comprising at least one of a polynomial function having at least one fit parameter, an exponential function having at least one fit parameter, a square root function having at least one fit parameter and a logarithmic function having at least one fit parameter. The DC signal S_{DC} may carry a non-zero power at at least one of the frequency f₀ and at least one overtone of the frequency f₀. For further details regarding the fitting, reference may be made to the definition of this term above.

Step c) comprises determining the AC signal S_{AC} by subtracting the DC signal S_{DC} from the measurement signal Sₘₑₐₛ by using the evaluation unit. The term “subtracting” including any grammatical variation thereof as used herein is a broad term and is to be given its ordinary and customary meaning to a person of ordinary skill in the art and is not to be limited to a special or customized meaning. The term specifically may refer, without limitation, to one or more of removing, eliminating and deducting the DC signal S_{DC} from the measurement signal Sₘₑₐₛ, specifically over an entire period of time. The subtracting may comprise extracting the AC signal S_{AC} from the measurement signal Sₘₑₐₛ. The subtracting may comprise at least one mathematical calculation. The subtracting may comprise subtracting the DC signal S_{DC}, such as a count of the DC signal S_{DC}, from the measurement signal Sₘₑₐₛ, such as from a count of the measurement signal Sₘₑₐₛ, specifically for each point in time individually over the entire period of time. In such way, the AC signal S_{AC} may for instance be determined for each point in time, thus for each chosen time unit, e.g. for each millisecond or for each frame number of a photodetector. Other options may be feasible.

The method for retrieving at least one AC signal S_{AC} from at least one measurement signal Sₘₑₐₛ may at least partially be computer-implemented. The term “computer-implemented” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the methods according to the present invention. Specifically, each of the method steps may be performed by the computer and/or computer network. The method may be performed completely automatically, specifically without user interaction.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

In a further aspect of the present invention, a method for determining at least one item of information on at least one measurement object by using at least one detector is disclosed. The method comprises the following steps:
i) determining at least one measurement signal Sₘₑₐₛ by using the detector;
ii) determining the AC signal S_{AC} by using a method according to any one of the embodiments disclosed above or below in further detail referring to a method for retrieving at least one AC signal S_{AC} from at least one measurement signal Sₘₑₐₛ; and
iii) determining the item of information on the measurement object by evaluating the AC signal S_{AC} by using the evaluation unit.

The method steps may be performed in the indicated order. It shall be noted, however, that a different order is also possible. The method may comprise further method steps which are not listed. Further, one or more of the method steps may be performed once or repeatedly. Further, two or more of the method steps may be performed simultaneously or in a timely overlapping fashion. For further definitions and embodiments regarding the method for determining at least one item of information, reference may be made to the description above.

The term “item of information” as used herein is a broad term and is to be given its ordinary and customary meaning to a person of ordinary skill in the art and is not to be limited to a special or customized meaning. The term specifically may refer, without limitation, to knowledge or evidence providing a qualitative and/or quantitative description relating to at least one measurement, specifically to the at least one measurement object. The item of information may comprise at least one of a physical property of the measurement object or a chemical composition of the at least one measurement object. The physical property may specifically comprise an optical property such at least one absorbance of the measurement object and/or at least one emissivity of the measurement object. The chemical composition may specifically refer to qualitative and/or quantitative information on at least one material the measurement object comprises.

The term “measurement object” as used herein is a broad term and is to be given its ordinary and customary meaning to a person of ordinary skill in the art and is not to be limited to a special or customized meaning. The term specifically may refer, without limitation, to an arbitrary body, chosen from a living body and a non-living body. The measurement object may specifically comprise at least one material which is subject to an investigation. The measurement object may generally refer to an object which is to be measured, e.g. for which a spectrum is to be recorded, wherein the measurement object may have in principle arbitrary properties, e.g. arbitrary optical properties or an arbitrary shape. The measurement object may comprise at least one solid sample. However, other measurement objects such as fluids may also be feasible.

The detector may comprise at least one photodetector. The photodetector may comprise at least one photosensitive region. Step i) may comprise measuring the measurement signal Sₘₑₐₛ by using the photosensitive region of the photodetector. The measurement signal Sₘₑₐₛ may be dependent on an illumination of the photosensitive region. For further definitions and embodiments regarding the photodetector, reference may be made to the description above.

The method for determining at least one item of information on at least one measurement object may at least partially be computer-implemented. Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

In a further aspect of the present invention, non-transient computer-readable medium is disclosed. The non-transient computer-readable medium includes instructions that, when executed by one or more processors, cause the one or more processors to perform at least one of the methods according to any one of the method embodiments disclosed above or below in further detail.

Specifically, the non-transient computer-readable medium includes instructions that, when executed by one or more processors, cause the one or more processors to perform the method for retrieving at least one alternating current (AC) signal S_{AC} from at least one measurement signal Sₘₑₐₛ of at least one detector according to the present invention. The measurement signal Sₘₑₐₛ comprises the AC signal S_{AC} and at least one direct current (DC) signal S_{DC}, wherein the AC signal S_{AC} has at least one predefined frequency f₀. In particular, the non-transient computer-readable medium includes instructions to cause the detector to monitor the measurement signal Sₘₑₐₛ over time. In particular, the non-transient computer-readable medium includes instructions to cause the evaluation unit to determine the DC signal S_{DC}, wherein the determining comprises evaluating the measurement signal Sₘₑₐₛ by using at least one of the frequency f₀ and at least one overtone of the frequency f₀. In particular, the non-transient computer-readable medium includes instructions to cause the evaluation unit to determine the AC signal S_{AC} by subtracting the DC signal S_{DC} from the measurement signal Sₘₑₐₛ.

The non-transient computer-readable medium may include instructions that, when executed by one or more processors, cause the one or more processors to perform the method for determining at least one item of information on at least one measurement object according to the present invention. In particular, the non-transient computer-readable medium includes instructions to cause the detector to determine at least one measurement signal Sₘₑₐₛ. In particular, the non-transient computer-readable medium includes instructions to cause determining the AC signal S_{AC} by using a method according to the present invention. In particular, the non-transient computer-readable medium includes instructions to cause the evaluation unit to determine the item of information on the measurement object by evaluating the AC signal S_{AC}.

In a further aspect of the present invention, a photodetector for measuring optical radiation is disclosed. The photodetector is configured for performing the method for retrieving at least one alternating current (AC) signal S_{AC} from at least one measurement signal Sₘₑₐₛ according to any one of the embodiments disclosed above or below in further detail referring to a method for retrieving at least one alternating current (AC) signal S_{AC} from at least one measurement signal Sₘₑₐₛ and/or for performing the method for determining at least one item of information on a measurement object according to any one of the embodiments disclosed above or below in further detail referring to a method for determining at least one item of information on a measurement object. The photodetector comprises at least one photosensitive region.

Specifically, the photodetector is configured for performing the method for retrieving at least one alternating current (AC) signal S_{AC} from at least one measurement signal Sₘₑₐₛ according to the present invention. In particular, the photodetector is configured for monitoring the measurement signal Sₘₑₐₛ over time. In particular, the photodetector comprises the evaluation unit configured for or may be connectable to at least one evaluation unit to determine the DC signal S_{DC}, wherein the determining comprises evaluating the measurement signal Sₘₑₐₛ by using at least one of the frequency f₀ and at least one overtone of the frequency f₀. The evaluation unit may be configured for determining the AC signal S_{AC} by subtracting the DC signal S_{DC} from the measurement signal Sₘₑₐₛ.

Specifically, the photodetector is configured for performing the method for determining at least one item of information on at least one measurement object according to the present invention. In particular, the photodetector is configured for determining at least one measurement signal Sₘₑₐₛ. In particular, the photodetector is configured for determining the AC signal S_{AC} by using a method according to the present invention. In particular, the photodetector comprises the evaluation unit configured for or may be connectable to at least one evaluation unit to determine the item of information on the measurement object by evaluating the AC signal S_{AC}.

The photodetector may be configured for detecting optical radiation in a wavelength of 300 nm to 3000 nm, specifically 500 nm to 2500 nm, more specifically 1400 nm to 2000 nm. The photosensitive region may comprise at least one photoconductive material. The photoconductive material may be selected from at least one of PbS, PbSe, Ge, InGaAs, InSb, and HgCdTe. Other options, such as photodiodes or thermopiles, may also be feasible. The photodetector may comprise at least one evaluation unit and/or at least one interface for transmitting data from and/or to and/or within the evaluation unit. The evaluation unit may at least partially be cloud based. For further definitions and embodiments regarding the photodetector, reference may be made to the description above.

In a further aspect of the present invention, a spectrometer for spectrally analyzing optical radiation provided by at least one measurement object is disclosed. The spectrometer comprises:
at least one radiation source configured for emitting optical radiation at least partially towards the measurement object; and
at least one photodetector according to any one of the embodiments disclosed above or below in further detail referring to a photodetector.

The term “spectrum” including an grammatical variation thereof as used herein is a broad term and is to be given its ordinary and customary meaning to a person of ordinary skill in the art and is not to be limited to a special or customized meaning. The term specifically may refer, without limitation, to a partition of the optical radiation, wherein the spectrum is constituted by an optical signal defined by a signal wavelength and a corresponding signal intensity. In particular, the spectrum may comprise spectral information related to at least one measurement object, such as a type and composition of at least one material forming the at least one measurement object, which can be determined by recording at least one spectrum related to the at least one measurement object. The term “spectrometer” as used herein is a broad term and is to be given its ordinary and customary meaning to a person of ordinary skill in the art and is not to be limited to a special or customized meaning. The term specifically may refer, without limitation, to an apparatus which is configured for determining spectral information by recording at least one measured value for at least one signal intensity related to at least one corresponding signal wavelength of optical radiation and by evaluating at least one measurement signal which relates to the signal intensity.

The spectrometer may further comprise at least one evaluation unit. The evaluation unit may further be configured for generating at least one item of spectral information on the measurement object. The evaluation unit may further be configured for controlling the radiation source, such as a modulation frequency of the radiation source. The spectrometer may further comprise at least one optical filter element. The optical filter element may be configured for filtering the optical radiation or more specifically selected wavelengths of the optical radiation. The at least one filter element may specifically be positioned in a beam path before at least one photosensitive region of the photodetector. The spectrometer may comprise a plurality of photosensitive regions and a plurality of optical filter elements, wherein at least one optical filter element may be positioned in a beam path before at least one photosensitive region, wherein the plurality of optical filter elements may be configured for at least partially filtering different wavelengths.

For further definitions and embodiments regarding the spectrometer, reference may be made to the description of the methods and devices above.

In a further aspect of the present invention, a use of a spectrometer according to any one of the embodiments disclosed above or below in further detail referring to a spectrometer is disclosed for a purpose of use, selected from the group consisting of: an infrared detection application; a heat detection application; a thermometer application; a heat-seeking application; a flame-detection application; a fire-detection application; a smoke-detection application; a temperature sensing application; a spectroscopy application; an exhaust gas monitoring application; a combustion process monitoring application; a pollution monitoring application; an industrial process monitoring application; a chemical process monitoring application; a food processing process monitoring application; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application, such as an animal feed application; a cosmetic application, such as a cosmetic application concerning hair.

Further disclosed and proposed herein is a computer program including computer-executable instructions for performing the methods according to the present invention in one or more of the embodiments enclosed herein when the program is executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps as indicated above may be performed by using a computer or a computer network, preferably by using a computer program.

Further disclosed and proposed herein is a computer program product having program code means, in order to perform the methods according to the present invention in one or more of the method embodiments enclosed herein when the program is executed on a computer or computer network. Specifically, the program code means may be stored on a computer-readable data carrier and/or on a computer-readable storage medium.

Further disclosed and proposed herein is a data carrier having a data structure stored thereon, which, after loading into a computer or computer network, such as into a working memory or main memory of the computer or computer network, may execute the methods according to one or more of the methods embodiments disclosed herein.

Further disclosed and proposed herein is a computer program product with program code means stored on a machine-readable carrier, in order to perform the methods according to one or more of the method embodiments disclosed herein, when the program is executed on a computer or computer network. As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Finally, disclosed and proposed herein is a modulated data signal which contains instructions readable by a computer system or computer network, for performing the methods according to one or more of the method embodiments disclosed herein.

Referring to the computer-implemented aspects of the invention, one or more of the method steps or even all of the method steps of the methods according to one or more of the method embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, further disclosed herein are:
a computer or computer network comprising at least one processor, wherein the processor is adapted to perform the methods according to one of the method embodiments described in this description,
a computer loadable data structure that is adapted to perform the methods according to one of the embodiments described in this description while the data structure is being executed on a computer,
a computer program, wherein the computer program is adapted to perform the methods according to one of the embodiments described in this description while the program is being executed on a computer,
a computer program comprising program means for performing the methods according to one of the method embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the methods according to one of the method embodiments described in this description after having been loaded into a main and/or working storage of a computer or of a computer network, and
a computer program product having program code means, wherein the program code means can be stored or are stored on a storage medium, for performing the methods according to one of the method embodiments described in this description, if the program code means are executed on a computer or on a computer network.

The methods and devices as disclosed herein have considerable advantages over the prior art. Specifically, the methods and devices disclosed herein may ensure an accurate and reliable compensation of drifts of detectors, specifically of temperature drifts of photodetectors, in a simple and safe fashion. They may rely purely on data analysis and thus avoid using additional components such as temperature sensors or other additional detectors for compensating the drift, which may typically reflect in cost, complexity and susceptibility to errors.

As used herein, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically are used only once when introducing the respective feature or element. In most cases, when referring to the respective feature or element, the expressions “at least one” or “one or more” are not repeated, nonwithstanding the fact that the respective feature or element may be present once or more than once.

Further, as used herein, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Simiarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

Summarizing and without excluding further possible embodiments, the following embodiments may be envisaged:

Embodiment 1: A method for retrieving at least one alternating current (AC) signal S_{AC} from at least one measurement signal Sₘₑₐₛ of at least one detector, wherein the measurement signal Sₘₑₐₛ comprises the AC signal S_{AC} and at least one direct current (DC) signal S_{DC}, wherein the AC signal S_{AC} has at least one predefined frequency f₀, the method comprising the following steps:
a) monitoring the measurement signal Sₘₑₐₛ over time by using the detector;
b) determining the DC signal S_{DC} by using at least one evaluation unit, wherein the determining comprises evaluating the measurement signal Sₘₑₐₛ by using at least one of the frequency f₀ and at least one overtone of the frequency f₀; and
c) determining the AC signal S_{AC} by subtracting the DC signal S_{DC} from the measurement signal Sₘₑₐₛ by using the evaluation unit.

Embodiment 2: The method according to the preceding embodiment, wherein the detector comprises at least one photodetector comprising at least one photosensitive region, wherein step a) comprises measuring the measurement signal Sₘₑₐₛ by using the photosensitive region of the photodetector, wherein the measurement signal Sₘₑₐₛ is dependent on an illumination of the photosensitive region.

Embodiment 3: The method according to the preceding embodiment, wherein the photodetector is configured for detecting optical radiation in a wavelength of 300 nm to 3000 nm, specifically 500 nm to 2500 nm, more specifically 1400 nm to 2000 nm.

Embodiment 4: The method according to any one of the two preceding embodiments, wherein the photosensitive region comprises at least one photoconductive material.

Embodiment 5: The method according to any one of the three preceding embodiments, wherein the photoconductive material is selected from at least one of PbS, PbSe, Ge, InGaAs, InSb, and HgCdTe.

Embodiment 6: The method according to any one of the preceding embodiments, wherein the method comprises correcting at least one environmental change affecting the measurement signal Sₘₑₐₛ, wherein the environmental change specifically comprises at least one of a temperature change, a change in a background light, a mechanical stress, a humidity change and a degradation of at least a part of the detector.

Embodiment 7: The method according to any one of the preceding embodiments, wherein in step b) the DC signal S_{DC} is determined by further using a phase φ of the measurement signal Sₘₑₐₛ, wherein the evaluation of the measurement signal Sₘₑₐₛ comprises determining local minima of the measurement signal Sₘₑₐₛ by using the phase φ and at least one of the frequency f₀ and at least one overtone of the frequency f₀, wherein the DC signal S_{DC} is determined by using the local minima.

Embodiment 8: The method according to the preceding embodiment, wherein the evaluation of the measurement signal Sₘₑₐₛ comprises fitting the DC signal S_{DC} to the local minima of the measurement signal Sₘₑₐₛ, wherein the DC signal S_{DC} is a function S_{DC}(t) over time comprising at least one of a polynomial function having at least one fit parameter, an exponential function having at least one fit parameter, a square root function having at least one fit parameter and a logarithmic function having at least one fit parameter.

Embodiment 9: The method according to any one of the preceding embodiments, wherein in step b) the DC signal S_{DC} is determined by transforming the measurement signal Sₘₑₐₛ into a frequency domain.

Embodiment 10: The method according to the preceding embodiment, wherein the measurement signal Sₘₑₐₛ is transformed into the frequency domain by using a Fourier transformation.

Embodiment 11: The method according to any one of the two preceding embodiments, wherein the evaluation of the measurement signal Sₘₑₐₛ comprises filtering the transformed measurement signal Sₘₑₐₛ for at least one of the frequency f₀ and at least one overtone of the frequency f₀.

Embodiment 12: The method according to the preceding embodiment, wherein the evaluation of the measurement signal Sₘₑₐₛ comprises using the filtered transformed measurement signal Sₘₑₐₛ for determining the DC signal S_{DC}.

Embodiment 13: The method according to the preceding embodiment, wherein the evaluation of the measurement signal Sₘₑₐₛ comprises fitting the DC signal S_{DC} to the filtered transformed measurement signal Sₘₑₐₛ, wherein the DC signal S_{DC} is a function S_{DC}(t) over time comprising at least one of a polynomial function having at least one fit parameter, an exponential function having at least one fit parameter, a square root function having at least one fit parameter and a logarithmic function having at least one fit parameter.

Embodiment 14: The method according to the preceding embodiment, wherein the DC signal S_{DC} carries a non-zero power at at least one of the frequency f₀ and at least one overtone of the frequency f₀.

Embodiment 15: The method according to any one of the preceding embodiments, wherein detector comprises the evaluation unit and/or at least one interface for transmitting data from and/or to and/or within the evaluation unit.

Embodiment 16: The method according to any one of the preceding embodiments, wherein the evaluation unit is at least partially cloud based.

Embodiment 17: The method according to anyone of the preceding method embodiments, wherein the method is at least partially computer-implemented.

Embodiment 18: A method for determining at least one item of information on at least one measurement object by using at least one detector, the method comprising the following steps:
i) determining at least one measurement signal Sₘₑₐₛ by using the detector;
ii) determining the AC signal S_{AC} by using a method according to any one of the preceding embodiments; and
iii) determining the item of information on the measurement object by evaluating the AC signal S_{AC} by using the evaluation unit.

Embodiment 19: The method according to the preceding embodiment, wherein the detector comprises at least one photodetector comprising at least one photosensitive region, wherein step i) comprises measuring the measurement signal Sₘₑₐₛ by using the photosensitive region of the photodetector, wherein the measurement signal Sₘₑₐₛ is dependent on an illumination of the photosensitive region.

Embodiment 20: The method according to any one of the preceding embodiments referring to a method for determining at least one item of information, wherein the method is at least partially computer-implemented.

Embodiment 21: A non-transient computer-readable medium including instructions that, when executed by one or more processors, cause the one or more processors to perform at least one of the methods according to any one of the preceding method embodiments.

Embodiment 22: A photodetector for measuring optical radiation, the photodetector being configured for performing the method for retrieving at least one alternating current (AC) signal S_{AC} from at least one measurement signal Sₘₑₐₛ according to any one of the preceding embodiments referring to a method for retrieving at least one alternating current (AC) signal S_{AC} from at least one measurement signal Sₘₑₐₛ and/or for performing the method for determining at least one item of information on a measurement object according to any one of the preceding embodiments referring to a method for determining at least one item of information on a measurement object, wherein the photodetector comprises at least one photosensitive region.

Embodiment 23: The photodetector according to the preceding embodiment, wherein the photodetector is configured for detecting optical radiation in a wavelength of 300 nm to 3000 nm, specifically 500 nm to 2500 nm, more specifically 1400 nm to 2000 nm.

Embodiment 24: The photodetector according to any one of the preceding embodiments referring to a photodetector, wherein the photosensitive region comprises at least one photoconductive material.

Embodiment 25: The photodetector according to the preceding embodiment, wherein the photoconductive material is selected from at least one of PbS, PbSe, Ge, InGaAs, InSb, and HgCdTe.

Embodiment 26: The photodetector according to any one of the preceding embodiments referring to a photodetector, wherein photodetector comprises at least one evaluation unit and/or at least one interface for transmitting data from and/or to and/or within the evaluation unit.

Embodiment 27: The photodetector according to any one of the preceding embodiments referring to a photodetector, wherein the evaluation unit is at least partially cloud based.

Embodiment 28: A spectrometer for spectrally analyzing optical radiation provided by at least one measurement object, the spectrometer comprising:
at least one radiation source configured for emitting optical radiation at least partially towards the measurement object; and
at least one photodetector according to any one of the preceding embodiments referring to a photodetector.

Embodiment 29: The spectrometer according to the preceding embodiment, wherein the spectrometer further comprises at least one evaluation unit, wherein the evaluation unit is further configured for generating at least one item of spectral information on the measurement object.

Embodiment 30: The spectrometer according to the preceding embodiment, wherein the evaluation unit is further configured for controlling the radiation source.

Embodiment 31: The spectrometer according to any one of the preceding embodiments referring to a spectrometer, wherein the radiation source is a modulated radiation source.

Embodiment 32: The spectrometer according to the preceding embodiment, wherein the radiation source is modulated at the frequency f₀.

Embodiment 33: The spectrometer according to any one of the preceding embodiments referring to a spectrometer, wherein the radiation source comprises at least one of a semiconductor-based radiation source, specifically at least one of a light emitting diode and a laser, and a thermal radiator, specifically an incandescent lamp.

Embodiment 34: The spectrometer according to any one of the preceding embodiments referring to a spectrometer, further comprising at least one optical filter element.

Embodiment 35: Use of a spectrometer according to any one of the preceding embodiments referring to a spectrometer for a purpose of use, selected from the group consisting of: an infrared detection application; a heat detection application; a thermometer application; a heat-seeking application; a flame-detection application; a fire-detection application; a smoke-detection application; a temperature sensing application; a spectroscopy application; an exhaust gas monitoring application; a combustion process monitoring application; a pollution monitoring application; an industrial process monitoring application; a chemical process monitoring application; a food processing process monitoring application; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application, such as an animal feed application; a cosmetic application, such as a cosmetic application concerning hair.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 schematically shows an exemplary embodiment of a spectrometer according to the present invention;

FIG. 2 schematically shows an exemplary embodiment of a photodetector according to the present invention;

FIG. 3 shows a flow chart of an exemplary embodiment of a method for retrieving at least one alternating current (AC) signal S_{AC} from at least one measurement signal Sₘₑₐₛ according to the present invention;

FIGS. 4-6B show experimental results of measurements on an exemplary embodiment of a spectrometer according to the present invention; and

FIG. 7 shows a flow chart of an exemplary embodiment of a method for determining at least one item of information on at least one measurement object according to the present invention.

### DETAILED DESCRIPTION OF THE EMBODIMENTS

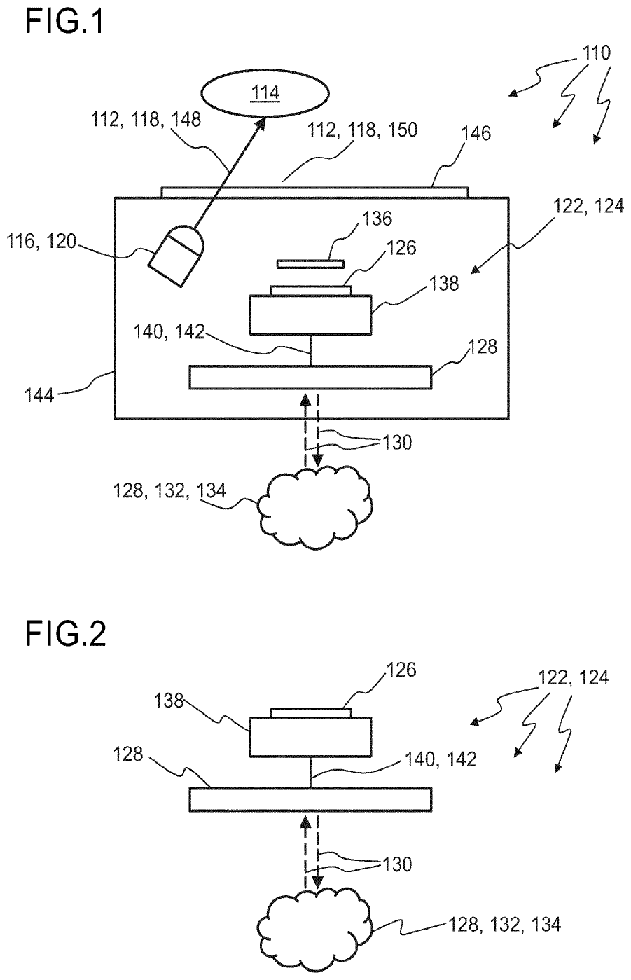

FIG. 1 schematically shows an exemplary embodiment of a spectrometer 110 according to the present invention. The spectrometer 110 is configured for spectrally analyzing optical radiation 112 provided by at least one measurement object 114. The optical radiation 112 may specifically be within at least one of the visible, the ultraviolet or the infrared spectral range. Preferably, the optical radiation 112 which is used for typical purposes of the present invention is IR radiation, more preferred, NIR radiation, especially of a wavelength of 760 nm to 3 μm, preferably of 1 μm to 3 μm. The optical radiation 112 may be provided by the measurement object 114. The providing may comprise at least one of a reflecting, transmitting and emitting. The measurement object 114 may be an arbitrary body, chosen from a living body and a non-living body. The measurement object 114 may specifically comprise at least one material which is subject to an investigation. The measurement object 114 may generally refer to an object which is to be measured, e.g. for which a spectrum is to be recorded, wherein the measurement object 114 may have in principle arbitrary properties, e.g. arbitrary optical properties or an arbitrary shape. The measurement object 114 may comprise at least one solid sample. However, other measurement objects such as fluids may also be feasible.

The spectrometer 110 may be an apparatus which is configured for determining spectral information by recording at least one measured value for at least one signal intensity related to at least one corresponding signal wavelength of the optical radiation 112 and by evaluating at least one measurement signal which relates to the signal intensity. The spectrometer 110 comprises at least one radiation source 116 configured for emitting the optical radiation 112 at least partially towards the measurement object 114. The radiation source 116 may be a device configured for emitting the optical radiation 112. The radiation source 116 may be configured for emitting the optical radiation 112 towards the measurement object 114, such as in form of a light beam 118. The radiation source 116 may be configured for isotopically emitting the optical radiation 112, e.g. uniformly in all spatial directions, wherein only a part of the emitted optical radiation 112 may impinge the measurement object 114. The radiation source 112 may comprise at least one of a semiconductor-based radiation source or a thermal radiator. The at least one semiconductor-based radiation source may be selected from at least one of a light emitting diode (LED) or a laser, specifically a laser diode. The LED may comprise at least one fluorescent and/or phosphorescent material. The thermal radiator may comprise at least one of an incandescent lamp, a black body emitter and a microelectromechanical system (MEMS) emitter.

The optical radiation 112 may be modulated, e.g. by using a modulated radiation source 120. In other words, the radiation source 116 may be a modulated radiation source 120. The radiation source 112 may be modulated at the frequency f₀. Thus, the frequency f₀ and overtones of the frequency f₀ may be present in the optical radiation 112. The modulating may be a process of changing, specifically periodically changing, at least one property of optical radiation, specifically one or both of an intensity or a phase of the optical radiation. The modulation may be a full modulation from a maximum value to zero, or may be a partial modulation, from a maximum value to an intermediate value greater than zero. The modulating may comprise using a modulating element. The modulating element may be configured for e.g. mechanically modulating the optical radiation, e.g. by using a rotating chopper wheel, and/or for electronically modulating the optical radiation, e.g. by using an electrooptic effect and/or an acoustoptic effect, e.g. by using a Pockels cell and/or a Kerr cell.

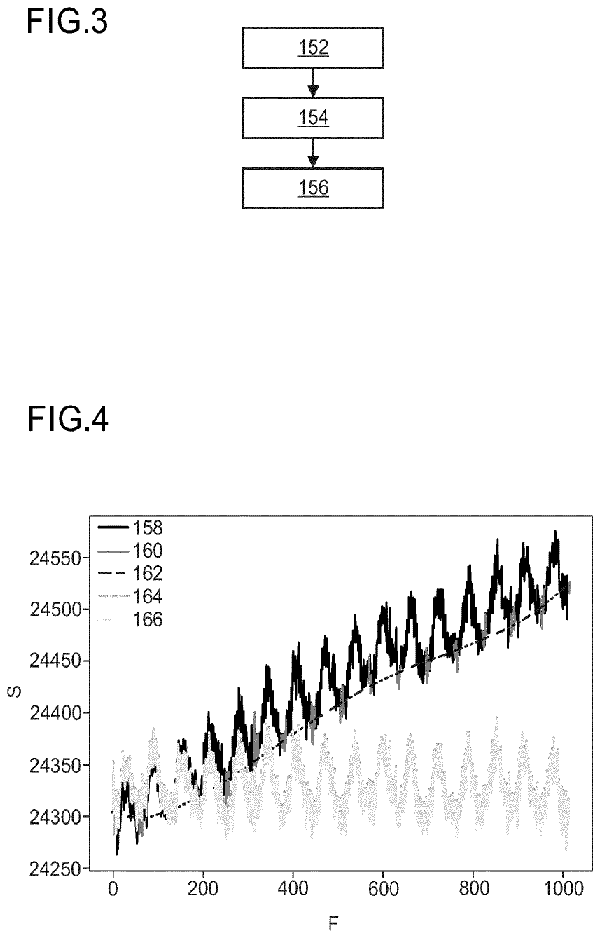

The spectrometer 110 comprises at least one photodetector 122 according to any one of the embodiments disclosed above or below in further detail referring to the photodetector 122. An exemplary embodiment of the photodetector 122 is also schematically shown in FIG. 2 in an isolated fashion. Thus, with respect to the photodetector 122, FIGS. 1 and 2 can be described in conjunction. Generally, the photodetector 122 is a specific type of a detector 124. The detector 124 may be a measurement device, such as a sensor, configured for generating at least one measurement signal. The detector 124 may be configured for sensing or detecting or monitoring at least one physical quantity. The detector 124 may be an electronic device or an optoelectronic device. The detector 124 may be configured for generating at least one electronic signal, such as a current or a voltage or a resistance. The detector 124 may be or comprise at least one photodetector 122. As already indicated, a plurality of types of detectors 124 are in principal conceivable in the context of the present invention. However, for illustration, the focus will be on the photodetector 122 in the following.

The photodetector 122 may be an optical detector or optical sensor configured for detecting the optical radiation 112, such as for detecting an illumination and/or a light spot generated by the at least one light beam 118. The photodetector 122 is configured for performing the method for retrieving at least one alternating current (AC) signal S_{AC} from at least one measurement signal Sₘₑₐₛ according to any one of the embodiments disclosed above or below in further detail referring to a method for retrieving at least one alternating current (AC) signal S_{AC} from at least one measurement signal Sₘₑₐₛ and/or for performing the method for determining at least one item of information on the measurement object 114 according to any one of the embodiments disclosed above or below in further detail referring to a method for determining at least one item of information on the measurement object 114. The photodetector 122 comprises at least one photosensitive region 126. The photosensitive region 126 may be a unit of the photodetector 122 configured for being illuminated, or in other words for receiving the optical radiation 112, and for generating at least one signal, such as an electronic signal, in response to the optical radiation 112. The photosensitive region 126 may be located on a surface of the photodetector 122. The photosensitive region 126 may specifically be a single, closed, uniform photosensitive region 126. The photosensitive region 126 may also be referred to as pixel P. The photodetector 126 may be configured for detecting the optical radiation 112 in a wavelength of 300 nm to 3000 nm, specifically 500 nm to 2500 nm, more specifically 1400 nm to 2000 nm. The photosensitive region 126 may comprise at least one photoconductive material. The photoconductive material may be selected from at least one of PbS, PbSe, Ge, InGaAs, InSb, and HgCdTe. Photodiodes or thermopiles may also be feasible.

The spectrometer 110 may comprise at least one evaluation unit 128. The evaluation unit 128 may be configured for generating at least one item of spectral information on the measurement object 114. The evaluation unit 128 may be configured for controlling the radiation source 116. Specifically, the evaluation unit 128 may be configured for controlling a modulation frequency of the modulated radiation source 120. The photodetector 122 may comprise the evaluation unit 128 and/or at least one interface 130 for transmitting data from and/or to and/or within the evaluation unit 128. The detector 124 may comprise the evaluation unit 128 and/or at least one interface 130 for transmitting data from and/or to and/or within the evaluation unit 128. The evaluation unit 128 may at least partially be cloud based. In other words, the at least one evaluation unit 128 may at least partially be distributed in at least one cloud 132 used for at least one of cloud computing or cloud storage. The cloud 132 may specifically comprise at least one external device 134, e.g. a computer or a computer network. The cloud 132 may refer to an outsourcing of the evaluation unit 128 or of parts of the evaluation unit 128 to at least partially interconnected external devices 134, specifically computers or computer networks having larger computing power and/or data storage volume. The external devices 134 may be arbitrarily spatially distributed. The external devices 134 may vary over time, specifically on demand. The external devices 134 may be interconnected by using the internet and/or at least one intranet.

The evaluation unit 128 may be a device configured for analyzing or interpreting data, specifically for determining at least one item of qualitative or quantitative information. The information may specifically be obtained by evaluating at least one signal, such as a signal generated by the detector, specifically the measurement signal Sₘₑₐₛ. The evaluation unit 128 may be or may comprise at least one of an integrated circuit, in particular an application-specific integrated circuit (ASIC), or a data processing device, in particular at least one of a digital signal processor (DSP), a field programmable gate array (FPGA), a microcontroller, a microcomputer, a computer, or an electronic communication unit, specifically a smartphone or a tablet. Further components may be feasible, in particular at least one preprocessing device or data acquisition device. The evaluation unit 128 may comprise the interface 130 or parts thereof. The interface 130 may in particular be wireless interface and/or wire-bound. The evaluation unit 128 can be designed to, completely or partially, control or drive further devices, such as the detector 124 or the photodetector 122. The evaluation unit 128 may be designed to carry out at least one measurement cycle in which a plurality of measurement signals may be picked up. The evaluation unit 128 may be designed to control the detector 124 or the photodetector 122 for performing at least one measurement and/or for generating at least one measurement signal.

Information as determined by the evaluation unit 128 may, in particular, be provided to at least one of a further apparatus, or to a user, preferably in at least one of an electronic, visual, acoustic, or tactile fashion. The information may be stored in at least one data storage unit, specifically in an internal data storage unit as comprised by the photodetector 122 or the detector 124, in particular by the at least one evaluation unit 128, or in an separate storage unit to which the information may be transmitted via the at least one interface 130. The separate storage unit may be comprised by the at least one electronic communication unit. The storage unit may in particular be configured for storing at least one electronic table, such as at least one look-up table.

The evaluation unit 128 may be configured to perform at least one computer program, in particular at least one computer program performing or supporting the step of generating the at information. By way of example, one or more algorithms may be implemented which, by using the at least one measurement signal as at least one input variable, may perform a transformation into a piece of information. For this purpose, the evaluation unit 128 may comprise at least one data processing device, in particular at least one of an electronic or an optical data processing device, which can be designed to generate the information by evaluating the at least one measurement signal. The evaluation unit 128 may be designed to use at least one measurement signal as at least one input variable and to generate the information by processing the at least one input variable. The processing can be performed in a consecutive, a parallel, or a combined manner. The evaluation unit 128 may use an arbitrary process for generating the information, in particular by calculation and/or using at least one stored and/or known relationship.

The interface 130 may be an item or element forming a boundary configured for transferring information. The interface 130 may specifically be a communication interface. In particular, the interface 130 may be configured for transferring information from a computational device, e.g. a computer, such as to send or output information, e.g. onto another device. Additionally or alternatively, the interface 130 may be configured for transferring information onto a computational device, e.g. onto a computer, such as to receive information. The interface 130 may specifically provide means for transferring or exchanging information. In particular, the interface 130 may provide a data transfer connection, e.g. Bluetooth, NFC, inductive coupling or the like. As an example, the interface 130 may be or may comprise at least one port comprising one or more of a network or internet port, a USB-port and a disk drive. The interface 130 may comprise at least one web interface.

The spectrometer 110 may comprise at least one optical filter element 136. The optical filter element 136 may be configured for filtering the optical radiation 112 or more specifically selected wavelengths of the optical radiation 112. The at least one filter element 136 may specifically be positioned in a beam path before at least one photosensitive region 126 of the photodetector 122. The spectrometer may comprise a plurality of photosensitive regions 126 and a plurality of optical filter elements 136, wherein at least one optical filter element 136 may be positioned in a beam path before at least one photosensitive region 126, wherein the plurality of optical filter elements 136 may be configured for at least partially filtering different wavelengths. The photodetector 122 may comprise at least one readout circuit 138. The readout circuit 138 may be configured for reading out at least one signal generated by the photosensitive region 126. The readout circuit 138 may be connected to further components of the photodetector 122, such as to at least one of the evaluation unit 128 or the interface 130, e.g. by using at least one wire 140 or at least one trace 142. The spectrometer 110 may comprise at least one housing 144 surrounding at least parts of the spectrometer 110, such as at least one of the radiation source 116 and the photodetector 122. The at least one external device 134 of cloud 132 may be arranged outside of the housing 144. The housing 144 may comprise at least one window 146. The window 146 may at least partially be transparent for the optical radiation 112.

In the following, an exemplary beam path of the optical radiation 112 is described with respect to FIG. 1. The at least one radiation emitting element 116 may emit the optical radiation 112 as incident optical radiation 148 through the window 146 towards the measurement object 114. The measurement object 114 may at least partially, specifically diffusely, reflect the incident optical radiation 148 towards the at least one photosensitive region 126 of the photodetector 122 in form of reflected optical radiation 150. The measurement object 114 may at least partially absorb the incident optical radiation 148, which may be indicative of at least one physical property or chemical composition of the measurement object 114. The reflected optical radiation 150 may pass the window 146 and the optical filter element 136 before reaching the photosensitive region 126. The photosensitive region 126 may generate a corresponding measurement signal Sₘₑₐₛ which may for instance be read out by using the readout circuit 138.

As already indicated, FIG. 2 schematically shows an exemplary embodiment of the photodetector 122 according to the present invention. For a description of the photodetector 122, it may largely be referred to the description of the spectrometer 110 above. As said, the photodetector 122 is configured for performing the method for retrieving at least one alternating current (AC) signal S_{AC} from at least one measurement signal Sₘₑₐₛ according to any one of the embodiments disclosed above or below in further detail referring to a method for retrieving at least one alternating current (AC) signal S_{AC} from at least one measurement signal Sₘₑₐₛ and/or for performing the method for determining at least one item of information on the measurement object 114 according to any one of the embodiments disclosed above or below in further detail referring to a method for determining at least one item of information on the measurement object 114.

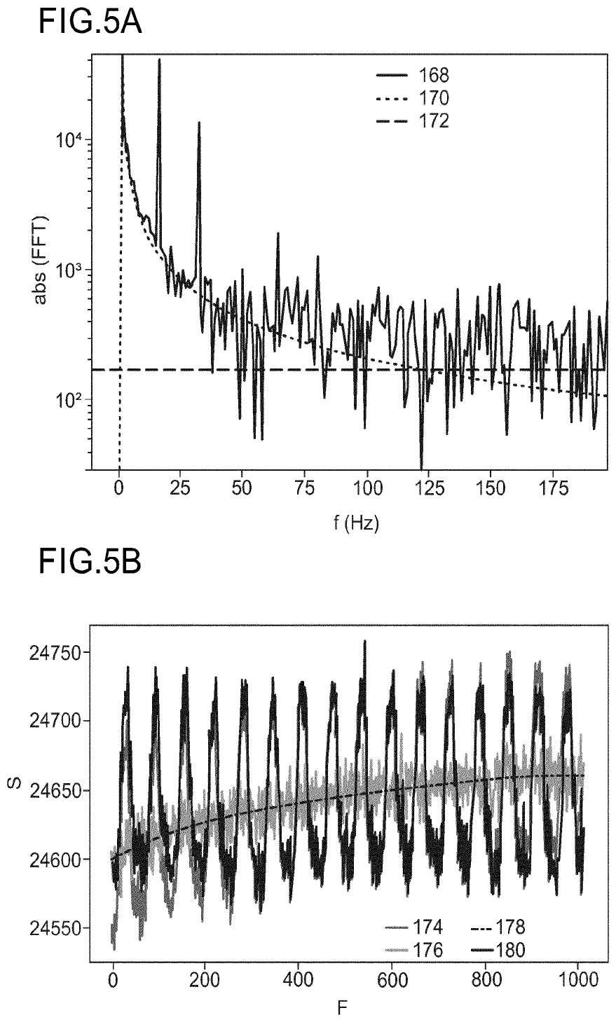

FIG. 3 shows a flow chart of an exemplary embodiment of a method for retrieving at least one alternating current (AC) signal S_{AC} from at least one measurement signal Sₘₑₐₛ of the detector 124. The measurement signal Sₘₑₐₛ comprises the AC signal S_{AC} and at least one direct current (DC) signal S_{DC}. The AC signal S_{AC} has at least one predefined frequency f₀. The method comprising the following steps:
a) (denoted with reference number 152) monitoring the measurement signal Sₘₑₐₛ over time by using the detector 124;
b) (denoted with reference number 154) determining the DC signal S_{DC} by using at least one evaluation unit 128, wherein the determining comprises evaluating the measurement signal Sₘₑₐₛ by using at least one of the frequency f₀ and at least one overtone of the frequency f₀; and
c) (denoted with reference number 156) determining the AC signal S_{AC} by subtracting the DC signal S_{DC} from the measurement signal Sₘₑₐₛ by using the evaluation unit 128.

The method steps may be performed in the indicated order. It shall be noted, however, that a different order is also possible. The method may comprise further method steps which are not listed herein. Further, one or more of the method steps may be performed once or repeatedly. Further, two or more of the method steps may be performed simultaneously or in a timely overlapping fashion. The method steps may at least partially be computer-implemented. As indicated, the detector 124 may comprise the photodetector 122 comprising at least one photosensitive region 126. Step a) may comprise measuring the measurement signal Sₘₑₐₛ by using the photosensitive region 126 of the photodetector 122. The measurement signal Sₘₑₐₛ may be dependent on an illumination of the photosensitive region 126.

The method may comprise correcting at least one environmental change affecting the measurement signal Sₘₑₐₛ. The environmental change may specifically comprise at least one of a temperature change, a change in a background light, a mechanical stress, and a humidity change and a degradation of at least a part of the detector. The correcting may be a compensating or a readjusting of an entity. The correcting may comprise removing or eliminating perturbations, specifically external perturbations, affecting the measurement signal Sₘₑₐₛ. Specifically, the correcting may comprise removing a contribution to the measurement signal Sₘₑₐₛ caused by an environmental change, such as a temperature change. Such a contribution may refer to a DC signal S_{DC}. As said, the detector 124 may be a photodetector 122 of a spectrometer 110 configured for measuring the optical radiation 112. Other external influences besides the optical radiation 112 to be measured may not be of interest in the measurement and may only disturb the measurement signal Sₘₑₐₛ. The spectrometer 110 may further comprise a modulated radiation source 120. Thus, the signal of interest may be an AC signal S_{AC}. External influences, e.g. temperature, may typically change on larger time scales compared to the AC signal S_{AC} and may be one-directional at least in the monitored period of time. The external influences may typically contribute in form of a DC signal S_{DC} to the measurement signal Sₘₑₐₛ. Identifying the DC signal S_{DC} in the measurement signal Sₘₑₐₛ and removing the DC signal S_{DC} from the measurement signal Sₘₑₐₛ may thus lead to the AC signal S_{AC} which may be of particular interest in the measurement.

The retrieving may be at least one of a determining, a deriving and a filtering out a signal or at least a part of the signal. As said, the measurement signal Sₘₑₐₛ comprises the AC signal S_{AC} and the DC signal S_{DC}. The retrieving may comprise identifying and/or isolating the AC signal S_{AC} in the measurement signal Sₘₑₐₛ. The retrieving may comprise removing and/or eliminating the DC signal S_{DC} from the measurement signal Sₘₑₐₛ. The retrieving may comprise providing the AC signal S_{AC} to further entities for further processing and/or evaluation, such as for determining an item of information, e.g. on the measurement object 114.

The signal may be an observable change in at least one physical quantity. The signal may be or comprise a sign or a function conveying information about the at least one physical quantity. The signal may specifically be or comprise at least one of an electronic signal, an optical signal or an optoelectronic signal. The signal may be a variable signal, specifically over time. The signal may be an analog signal. The signal may be or comprise at least one of a variable voltage, a variable current, a variable charge, a variable resistance or, generally, a variable electromagnetic wave. The variable electromagnetic wave may comprise at least one of a variable amplitude, a variable frequency or a variable phase. The signal may be a digital signal. The signal may comprise at least one count. The signal may specifically be related to at least one measurement. The signal may specifically be generated by the detector 124.

The measurement signal may be a signal relating to at least one measurement, more specifically to the measurement object 114. The measurement signal may be a signal generated by the detector 124 upon detection of at least one physical quantity, such as a physical quantity of the measurement object 114. The measurement signal may comprise at least one electronic signal, such as a current or a voltage or a resistance. The measurement signal may comprise an analog signal. The measurement signal may comprise a digital signal, such as a count. The measurement signal may be a superposition of two or more signals or sub-signals. The measurement may be affected by plurality of influences, such as illumination, temperature, humidity or mechanical stress. Each influence may contribute to the measurement signal. The measurement signal may be dividable into two or more sub-signals, wherein the sub-signals may at least partially relate to different influences.

The DC signal S_{DC} may be a one-directional or at least essentially one-directional signal over time, such as a continuously increasing signal over time or a continuously decreasing signal over time. As an example, the DC signal S_{DC} may be a digital signal, wherein a count may continuously increases over time. The DC signal S_{DC} may comprise at least one plateau over the course of time. Deviations from a strictly one-directional progression may e.g. arise due to signal noise or external perturbations.

The AC signal S_{AC} may be a signal which over time reverses direction and/or changes its magnitude, e.g. periodically. As an example, the AC signal S_{AC} may be a digital signal, wherein a count increases and decreases over time in an alternating fashion. The AC signal S_{AC} may be a sinusoidal signal, a square wave, a pulse-width modulated signal, or a combination of the previously mentioned ones. The AC signal S_{AC} may be a periodic signal or an at least essentially periodic signal. Deviations from a strictly periodic progression may e.g. arise due to signal noise or external perturbations. As said, the AC signal S_{AC} has at least one predefined frequency f₀. The frequency may generally be a number of occurrences of a repeating event over time. The frequency can be defined as a reciprocal of a period duration, such as a period duration of a periodic signal. The frequency may be predefined by at least one default, such as at least one default in a measurement setup. A user may be allowed to set the default or to choose between a number of different available defaults. As said, the detector 124 may be a photodetector 122 of a spectrometer 110, wherein the spectrometer 110 may further comprise the modulated radiation source 120. Thus, the frequency of the AC signal S_{AC} may be predefined by setting a specific modulation frequency at the modulated radiation source 120. An overtone may be a harmonic of a fundamental frequency, such as of the frequency f₀. An overtone of the frequency f₀ may be a positive integer multiple of the frequency f₀, such as 2 f₀, 3 f₀, 4 f₀ and so on.

Step a) comprises monitoring the measurement signal Sₘₑₐₛ over time by using the detector 124. The monitoring over time may be at least one of measuring, observing or recording an entity, such as the measurement signal Sₘₑₐₛ, specifically over time. The monitoring may comprise recording a progression and/or a development of the measurement signal Sₘₑₐₛ over time.

Step b) comprises determining the DC signal S_{DC} by using the evaluation unit 128, wherein the determining comprises evaluating the measurement signal Sₘₑₐₛ by using at least one of the frequency f₀ and at least one overtone of the frequency f₀. The evaluating may be a processing or an analyzing or an interpreting of an entity, such as of the measurement signal Sₘₑₐₛ. The evaluating may comprise performing at least one mathematical calculation involving the measurement signal Sₘₑₐₛ. The evaluating may comprise transforming and/or converting the measurement signal Sₘₑₐₛ. The evaluating may comprise using at least one relationship, such as a predefined and/or predetermined relationship, e.g. from a look-up table, or a variable relationship, such as function. The evaluating may comprise filtering and/or smoothening the measurement signal Sₘₑₐₛ. The evaluating may comprise deriving at least one qualitative or quantitative item of information from the measurement signal Sₘₑₐₛ, such as a contribution of the DC signal S_{DC} and/or the AC signal S_{AC} to the measurement signal Sₘₑₐₛ. Different approaches may be possible for such purpose as will be outlined in further detail below.

Step c) comprises determining the AC signal S_{AC} by subtracting the DC signal S_{DC} from the measurement signal Sₘₑₐₛ by using the evaluation unit. The subtracting may be one or more of removing, eliminating and deducting the DC signal S_{DC} from the measurement signal Sₘₑₐₛ, specifically over an entire period of time. The subtracting may comprise extracting the AC signal S_{AC} from the measurement signal Sₘₑₐₛ. The subtracting may comprise at least one mathematical calculation. The subtracting may comprise subtracting the DC signal S_{DC}, such as a count of the DC signal S_{DC}, from the measurement signal Sₘₑₐₛ, such as from a count of the measurement signal Sₘₑₐₛ, specifically for each point in time individually over the entire period of time. In such way, the AC signal S_{AC} may for instance be determined for each point in time, thus for each chosen time unit, e.g. for each millisecond or for each frame number of the photodetector 122.

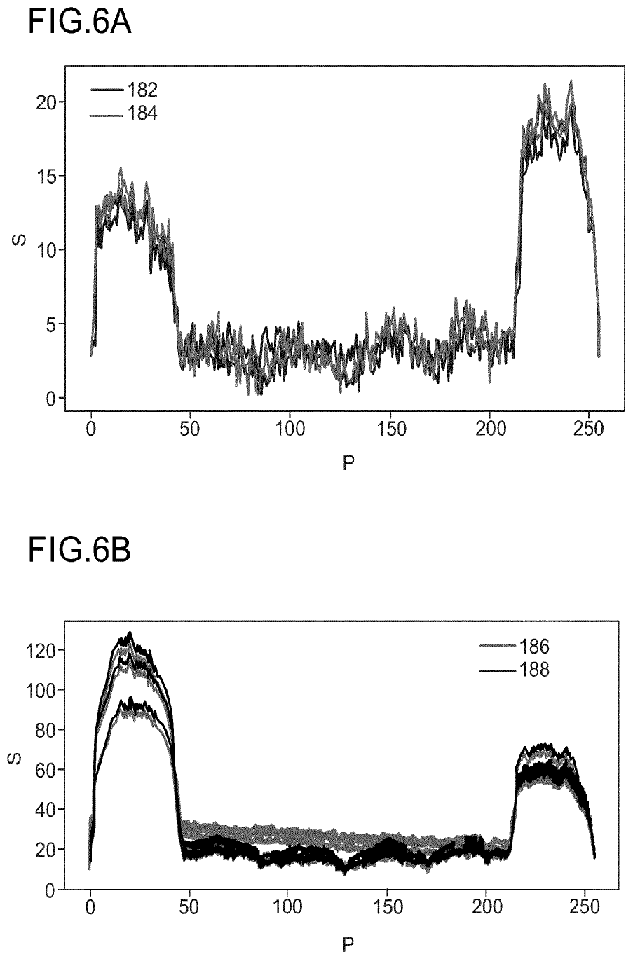

FIGS. 4-6B show experimental results of measurements on an exemplary embodiment of a spectrometer 110 according to the present invention. FIG. 4 shows a first approach for determining the DC signal S_{DC} in step b), wherein signals S are counted over frames F. A raw measurement signal is denoted with reference number 158. A masked measurement signal is denoted with reference number 160. In FIG. 4, the masked measurement signal 160 masks times when the AC signal S_{AC} reaches local minima, specifically based on at least one of the frequency f₀, at least one overtone of the frequency f₀ and a phase φ. The DC signal S_{DC} may be determined by using a phase φ of the measurement signal Sₘₑₐₛ, specifically of the AC signal S_{AC}, specifically besides the frequency f₀. The evaluation of the measurement signal Sₘₑₐₛ may comprise determining local minima of the measurement signal Sₘₑₐₛ by using the frequency f₀ and the phase φ and at least one of the frequency f₀ and at least one overtone of the frequency f₀. The DC signal S_{DC} may be determined by using the local minima. The phase may be a position indicator within a periodic signal. Typically, the phase may be represented as an angle. The phase may be dependent on the time, on a frequency of the signal and/or on a phase offset. Specifically, the phase may indicate when a periodic signal or a periodic part of a total signal, such as a periodic sub-signal, reaches an extremum, such as a minimum or a maximum. As an example, the measurement signal Sₘₑₐₛ may show an at least essentially periodic sub-signal, e.g. due to using a modulated radiation source as already outlined. This sub-signal may correspond to the AC signal S_{AC} which may be of interest in the end, but which at this stage may drift e.g. due to an environmental change. The frequency f₀ and the phase φ of this sub-signal may be used for identifying minima in the sub-signal, which may simultaneously at least be local minima in the overall measurement signal Sₘₑₐₛ. The local minimum may be a lowest signal value in a signal interval, specifically within a period of a periodic signal. The signal may comprise a plurality of local minima. The local minima may at least partially have the same level. The local minima may specifically at least partially have different levels, specifically due to external perturbations, such as an environmental change affecting the detector and/or the measurement signal. The measurement signal Sₘₑₐₛ may specifically be recorded in counts over time. As said, the measurement signal comprises the AC signal S_{AC} and the DC signal S_{DC}. A local minimum may be a lowest count in a time interval relating to a period of the AC signal S_{AC}.

The evaluation of the measurement signal Sₘₑₐₛ may comprise fitting the DC signal S_{DC} to the local minima of the measurement signal Sₘₑₐₛ. The DC signal S_{DC} may be a function S_{DC}(t) over time comprising at least one of a polynomial function having at least one fit parameter, an exponential function having at least one fit parameter, a square root function having at least one fit parameter and a logarithmic function having at least one fit parameter. The fitting may comprise a regression analysis for estimating a relation between at least two variables. The fitting may comprise at least one of a linear regression, a partial least square regression, a non-linear regression, an interpolation and an extrapolation. The fitting may comprise at least one regression model, e.g. a trained model. The fitting may comprise using at least one fit function, such as at least one of the functions listed above. The function S_{DC}(t) may be a fit function fitted to the local minima of the measurement signal Sₘₑₐₛ. The fit function may comprise at least one fit parameter. The fit parameter may be a parameter or a coefficient of a fit function. As an example, the fit function may be a linear function and the fit parameters may be a slope and an offset of the linear function.

As specifically shown in FIG. 4, a 4^{th} order polynomial function may be fitted to the masked measurement signal 160. The fitted baseline is denoted with reference number 162. The fitted baseline 162 may be subtracted from the raw measurement signal 158 for determining the AC signal S_{AC}. The in such way obtained corrected measurement signal based on the masked measurement signal is denoted with reference sign 164. Additionally or alternatively, as will be outlined in further detail below, the measurement signal may be corrected by using frequency filtering. The in such way obtained corrected measurement signal by using frequency filtering is denoted with reference sign 166. As FIG. 4 indicates, both approaches show comparable performance.

FIGS. 5A and 5B show a further approach for determining the DC signal S_{DC} in step b). Additionally or alternatively to the above-described approach using the local minima of the measurement signal Sₘₑₐₛ, in step b) the DC signal S_{DC} may be determined by transforming the measurement signal Sₘₑₐₛ into a frequency domain. The frequency domain may refer to an analysis of a signal with respect to at least one frequency of the signal. A signal may typically be recorded over time in the time domain meaning that a signal value is related to a specific point in time. However, for a variety of applications, it may be helpful to analyze the signal with respect to frequencies comprised by the signal in the frequency domain. As an example, one total signal may comprise a plurality of sub-signals each comprising a specific frequency. The sub-signals may be distinguishable, e.g. for further isolated processing, by analyzing the frequencies in the total signal. In the frequency domain, a signal value may be related to a specific frequency. Signal values, such as signal values of a total signal, may be plotted over a frequency interval in the frequency domain.

The measurement signal Sₘₑₐₛ may be transformed into the frequency domain by using a Fourier transformation. FIG. 5A shows such Fourier transformed signals obtained by using fast Fourier transformation (FFT), wherein the absolute signal counts are plotted on a logarithmic scale over the corresponding frequency f. The Fourier transformed raw measurement signal is denoted with reference number 168. The frequency f₀ and the first overtone at 2 f₀ are recognizable as peaks at approximately 15 Hz and 30 Hz, respectively. A distortion approximation is fitted to the Fourier transformed raw measurement signal 168 in FIG. 5A and is denoted with reference number 170. Further, a noise floor is denoted with reference number 172. The Fourier transformation may be an integral transformation for decomposing an integratable function depending on space or time into a function depending on spatial frequency or temporal frequency. Specifically, the Fourier transformation may be configured for decomposing a time dependent signal into a frequency dependent signal. The Fourier transformation may comprise at least one of a Fourier analysis, a continuous Fourier transformation, a discrete Fourier transformation and a Fourier related transformation, such as a Laplace transformation for instance. In step b), the DC signal S_{DC} may specifically be determined by setting at least one of the frequency f₀ and at least one overtone of the frequency f₀ to zero in the frequency domain before retransformation into the time domain, such as by using an inverse Fourier transformation. Thus, step c) may specifically be performed in the time domain again.

The evaluation of the measurement signal Sₘₑₐₛ, such as performed in step b), may comprise filtering the transformed measurement signal Sₘₑₐₛ for at least one of the frequency f₀ and at least one overtone of the frequency f₀. The filtering may be a selectively extracting at least one part of a signal, such as a specific signal interval, e.g. in the frequency domain. The filtering may comprise extracting specific frequencies, or sub-signals having specific frequencies, out of a total signal in the frequency domain. Specifically, the filtering may comprise extracting the at least one of the frequency f₀ and at least one overtone of the frequency f₀ from the measurement signal Sₘₑₐₛ. The rest of the original measurement signal Sₘₑₐₛ may stay as a remainder. The frequency f₀ and overtones of the frequency f₀ may refer to the AC signal S_{AC}. The remainder may refer to the DC signal S_{DC}. The filtering may comprise using at least one electronic filter element, specifically at least one frequency electronic filter element, such as an electronic bandpass filter element. The electronic filter element may be an analog electronic filter element or a digital electronic filter element.

The evaluation of the measurement signal Sₘₑₐₛ may comprise using the filtered transformed measurement signal Sₘₑₐₛ for determining the DC signal S_{DC}. Specifically, the evaluation of the measurement signal Sₘₑₐₛ may comprise using a remainder of the filtering of the transformed measurement signal Sₘₑₐₛ for determining the DC signal S_{DC} The evaluation of the measurement signal Sₘₑₐₛ may comprise fitting the DC signal S_{DC} to the filtered transformed measurement signal Sₘₑₐₛ, specifically to the remainder of the filtering of the transformed measurement signal Sₘₑₐₛ. The DC signal S_{DC} may be a function S_{DC}(t) over time comprising at least one of a polynomial function having at least one fit parameter, an exponential function having at least one fit parameter, a square root function having at least one fit parameter and a logarithmic function having at least one fit parameter. The DC signal S_{DC} may carry a non-zero power at at least one of the frequency f₀ and at least one overtone of the frequency f₀. FIG. 5B shows signals S counted over frames F in the time domain. A raw measurement signal is denoted with reference number 174. A filtered measurement signal, wherein a carrier frequency and overtones are filtered out, is denoted with reference number 176. A fitted correction is denoted with reference number 178 and a corrected measurement signal is denoted with reference number 180.

FIGS. 6A and 6B show applications of the method for retrieving at least one alternating current (AC) signal S_{AC} from at least one measurement signal Sₘₑₐₛ for different conditions. FIG. 6A shows a correction of a signal S for the case S_{DC}<<S_{AC} for 250 pixels P. A raw fast Fourier transformed (FFT) measurement signal is denoted with reference number 182. A corrected fast Fourier transformed (FFT) measurement signal is denoted with reference number 184. FIG. 6B shows a correction of a signal S for the cases S_{DC}˜S_{AC} or S_{DC}>>S_{AC} for 250 pixels P. A raw fast Fourier transformed (FFT) measurement signal is denoted with reference number 186. A corrected fast Fourier transformed (FFT) measurement signal is denoted with reference number 188. For all conditions, a clear signal recovery is evident.

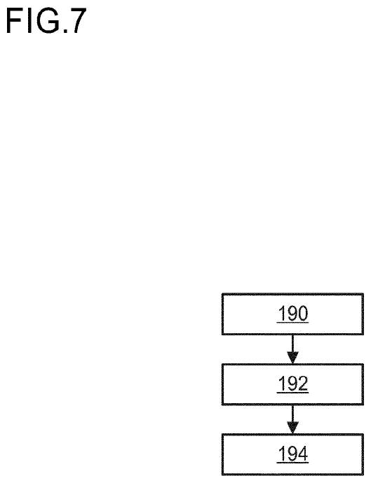

FIG. 7 shows a flow chart of an exemplary embodiment of a method for determining at least one item of information on the measurement object 114 by using the detector 124. The method comprises the following steps:
i) (denoted with reference number 190) determining at least one measurement signal Sₘₑₐₛ by using the detector 124;
ii) (denoted with reference number 192) determining the AC signal S_{AC} by using a method according to any one of the embodiments disclosed above or below in further detail referring to a method for retrieving at least one AC signal S_{AC} from at least one measurement signal Sₘₑₐₛ; and
iii) (denoted with reference number 194) determining the item of information on the measurement object 114 by evaluating the AC signal S_{AC} by using the evaluation unit 128.

The method steps may be performed in the indicated order. It shall be noted, however, that a different order is also possible. The method may comprise further method steps which are not listed herein. Further, one or more of the method steps may be performed once or repeatedly. Further, two or more of the method steps may be performed simultaneously or in a timely overlapping fashion. The method steps may at least partially be computer-implemented. The item of information may be knowledge or evidence providing a qualitative and/or quantitative description relating to at least one measurement, specifically to the at least one measurement object 114. The item of information may comprise at least one of a physical property of the measurement 114 object or a chemical composition of the at least one measurement object 114. The physical property may specifically comprise an optical property such at least one absorbance of the measurement object 114 and/or at least one emissivity of the measurement object 114. The chemical composition may specifically refer to qualitative and/or quantitative information on at least one material the measurement object 114 comprises.

### LIST OF REFERENCE NUMBERS

110 spectrometer
112 optical radiation
114 measurement object
116 radiation source
118 light beam
120 modulated radiation source
122 photodetector
124 detector
126 photosensitive region
128 evaluation unit
130 interface
132 cloud
134 external device
136 optical filter element
138 readout circuit
140 wire
142 trace
144 housing
146 window
148 incident optical radiation
150 reflected optical radiation
152 method step a)
154 method step b)
156 method step c)
158 raw measurement signal
160 masked measurement signal
162 fitted baseline
164 corrected measurement signal based on the masked measurement signal
166 corrected measurement signal based on using frequency filtering
168 Fourier transformed raw measurement signal
170 distortion approximation
172 noise floor
174 raw measurement signal
176 filtered measurement signal
178 fitted correction
180 corrected measurement signal
182 raw fast Fourier transformed (FFT) measurement signal
184 corrected fast Fourier transformed (FFT) measurement signal
186 raw fast Fourier transformed (FFT) measurement signal
188 corrected fast Fourier transformed (FFT) measurement signal
190 method step i)
192 method step ii)
194 method step iii)

1. A method for retrieving at least one alternating current (AC) signal S_{AC} from at least one measurement signal Sₘₑₐₛ of at least one detector, wherein the measurement signal Sₘₑₐₛ comprises the AC signal S_{AC} and at least one direct current (DC) signal S_{DC}, wherein the AC signal S_{AC} has at least one predefined frequency f₀, the method comprising the following steps:
a) monitoring the measurement signal Sₘₑₐₛ over time by using the detector;
b) determining the DC signal S_{DC} by using at least one evaluation unit, wherein the determining comprises evaluating the measurement signal Sₘₑₐₛ by using at least one of the frequency f₀ and at least one overtone of the frequency f₀; and
c) determining the AC signal S_{AC} by subtracting the DC signal S_{DC} from the measurement signal Sₘₑₐₛ by using the evaluation unit.

2. The method according to claim 1, wherein the detector comprises at least one photodetector comprising at least one photosensitive region, wherein step a) comprises measuring the measurement signal Sₘₑₐₛ by using the photosensitive region of the photodetector, wherein the measurement signal Sₘₑₐₛ is dependent on an illumination of the photosensitive region.

3. The method according to claim 1, wherein in step b) the DC signal S_{DC} is determined by further using a phase φ of the measurement signal Sₘₑₐₛ, wherein the evaluation of the measurement signal Sₘₑₐₛ comprises determining local minima of the measurement signal Sₘₑₐₛ by using the phase φ and at least one of the frequency f₀ and at least one overtone of the frequency f₀, wherein the DC signal S_{DC} is determined by using the local minima.

4. The method according to claim 3, wherein the evaluation of the measurement signal Sₘₑₐₛ comprises fitting the DC signal S_{DC} to the local minima of the measurement signal Sₘₑₐₛ, wherein the DC signal S_{DC} is a function S_{DC}(t) over time comprising at least one of a polynomial function having at least one fit parameter, an exponential function having at least one fit parameter, a square root function having at least one fit parameter and a logarithmic function having at least one fit parameter.

5. The method according to claim 1, wherein in step b) the DC signal S_{DC} is determined by transforming the measurement signal Sₘₑₐₛ into a frequency domain, wherein the measurement signal Sₘₑₐₛ is transformed into the frequency domain by using a Fourier transformation.

6. The method according to claim 1, wherein the evaluation of the measurement signal Sₘₑₐₛ comprises filtering the transformed measurement signal Sₘₑₐₛ for at least one of the frequency f₀ and at least one overtone of the frequency f₀, wherein the evaluation of the measurement signal Sₘₑₐₛ comprises using the filtered transformed measurement signal Sₘₑₐₛ for determining the DC signal S_{DC}.

7. The method according to claim 6, wherein the evaluation of the measurement signal Sₘₑₐₛ comprises fitting the DC signal S_{DC} to the filtered transformed measurement signal Sₘₑₐₛ, wherein the DC signal S_{DC} is a function S_{DC}(t) over time comprising at least one of a polynomial function having at least one fit parameter, an exponential function having at least one fit parameter, a square root function having at least one fit parameter and a logarithmic function having at least one fit parameter.

8. The method according to claim 1, wherein the detector comprises the evaluation unit and/or at least one interface for transmitting data from and/or to and/or within the evaluation unit, wherein the evaluation unit is at least partially cloud based.

9. The method according to claim 1, wherein the method is at least partially computer-implemented.

10. A method for determining at least one item of information on at least one measurement object by using at least one detector, the method comprising the following steps:
i) determining at least one measurement signal Sₘₑₐₛ by using the detector;
ii) determining the AC signal S_{AC} by using the method according to claim 1; and
iii) determining the item of information on the measurement object by evaluating the AC signal S_{AC} by using the evaluation unit (128).

11. A non-transient computer-readable medium including instructions that, when executed by one or more processors, cause the one or more processors to perform the method according to claim 1.

12. A photodetector for measuring optical radiation, the photodetector being configured for performing the method according to claim 1, wherein the photodetector comprises at least one photosensitive region.

13. A spectrometer for spectrally analyzing optical radiation provided by at least one measurement object, the spectrometer comprising:
at least one radiation source configured for emitting optical radiation at least partially towards the measurement object; and
at least one photodetector according to claim 12.

14. The spectrometer according to claim 13, wherein the radiation source is a modulated radiation source, wherein the radiation source is modulated at the frequency f₀.

15. A method of using the spectrometer according to claim 13, the method comprising using the spectrometer for a purpose of use selected from the group consisting of: an infrared detection application; a heat detection application; a thermometer application; a heat-seeking application; a flame-detection application; a fire-detection application; a smoke-detection application; a temperature sensing application; a spectroscopy application; an exhaust gas monitoring application; a combustion process monitoring application; a pollution monitoring application; an industrial process monitoring application; a chemical process monitoring application; a food processing process monitoring application; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application; and a cosmetic application.

16. A non-transient computer-readable medium including instructions that, when executed by one or more processors, cause the one or more processors to perform the method according to claim 10.

17. A photodetector for measuring optical radiation, the photodetector being configured for performing the method according to claim 10, wherein the photodetector comprises at least one photosensitive region.

18. A spectrometer for spectrally analyzing optical radiation provided by at least one measurement object, the spectrometer comprising:
at least one radiation source configured for emitting optical radiation at least partially towards the measurement object; and
at least one photodetector according to claim 17.

19. The spectrometer according to claim 18, wherein the radiation source is a modulated radiation source, wherein the radiation source is modulated at the frequency f₀.

20. A method of using the spectrometer according to claim 18, the method comprising using the spectrometer for a purpose of use selected from the group consisting of: an infrared detection application; a heat detection application; a thermometer application; a heat-seeking application; a flame-detection application; a fire-detection application; a smoke-detection application; a temperature sensing application; a spectroscopy application; an exhaust gas monitoring application; a combustion process monitoring application; a pollution monitoring application; an industrial process monitoring application; a chemical process monitoring application; a food processing process monitoring application; a water quality monitoring application; an air quality monitoring application; a quality control application; a temperature control application; a motion control application; an exhaust control application; a gas sensing application; a gas analytics application; a motion sensing application; a chemical sensing application; a mobile application; a medical application; a mobile spectroscopy application; a food analysis application; an agricultural application; and a cosmetic application.
